(19) United States
(12) Patent Application Publication
(10) Pub. No.: US 2025/0288310 A1
(43) Pub. Date: Sep. 18, 2025

(54) TRANSFER INSTRUMENT

(71) Applicant: TERUMO KABUSHIKI KAISHA, Tokyo (JP)

(72) Inventor: Koichi Hayakawa, Ashigarakami-gun (JP)

(73) Assignee: TERUMO KABUSHIKI KAISHA, Tokyo (JP)

(21) Appl. No.: 19/222,002

(22) Filed: May 29, 2025

Related U.S. Application Data
(63) Continuation of application No. PCT/JP2023/043041, filed on Dec. 1, 2023.

(30) Foreign Application Priority Data
Dec. 2, 2022 (JP) 2022-0193739

(51) Int. Cl. A61B 17/28

(52) U.S. Cl. CPC A61B 17/28

(57) ABSTRACT

A transfer instrument includes a first shaft having a tubular shape, a second shaft movably inserted through the first shaft, and a liquid supply portion capable of supplying a liquid toward a first support surface of a first support portion via a distal-end opening of the first shaft.

### CROSS-REFERENCES TO RELATED APPLICATIONS

This application is a continuation of International Application No. PCT/JP2023/043041 filed on Dec. 1, 2023, which claims priority to Japanese Application No. 2022-0193739 filed on Dec. 2, 2022, the entire content of both of which is incorporated herein by reference.

### TECHNOLOGICAL FIELD

The present disclosure generally relates to a transfer instrument for transferring a medical sheet to a treatment site of a living body.

### BACKGROUND DISCUSSION

Japanese Patent Application Publication No. 2009-000511 A discloses a transfer instrument for transferring a medical sheet (cell sheet) for use in, for example, organ transplantation to a treatment site of a living body. The transfer instrument includes a shaft and a support portion provided at a distal portion of the shaft. The support portion includes a sheet support on which the medical sheet is placed. The transfer instrument is slid while pressing an upper surface of the medical sheet placed on a support surface of the sheet support with forceps or the like to transfer the medical sheet from the support portion to the treatment site.

In the above-described related art, when the medical sheet is transferred from the support surface of the support portion to the treatment site of the living body, it is necessary to manipulate the transfer instrument while pressing the upper surface of the medical sheet with forceps or the like, which raises a concern that the medical sheet cannot be efficiently transferred to the treatment site.

### SUMMARY

(1) An aspect of the present disclosure is a transfer instrument for transferring a medical sheet to a treatment site of a living body, the transfer instrument including: an outer cylinder; a first carrier member including a first shaft that has a tubular shape, extends in an axial direction of the outer cylinder, and is disposed inside the outer cylinder to be movable along the axial direction, and a support portion that has a sheet shape, is disposed at a distal portion of the first shaft, and includes a support surface capable of holding the medical sheet when the distal portion of the first shaft is disposed in a distal end direction with respect to a distal portion of the outer cylinder; a second carrier member including a second shaft inserted through the first shaft, in which the second shaft extends along the first shaft and is provided movably along the first shaft; and a liquid supply portion capable of supplying a liquid into the first shaft in order to supply the liquid toward the support surface through a distal-end opening of the first shaft, in which, by moving the first shaft and the second shaft in a proximal end direction with respect to the outer cylinder such that the support portion is retracted into the outer cylinder, the support portion is retracted into the outer cylinder in a state of being curved and deformed, and by moving the first shaft and the second shaft in the distal end direction with respect to the outer cylinder such that the support portion protrudes from a distal-end opening of the outer cylinder, the support portion is unfolded by being exposed from the outer cylinder in the distal end direction.

Since the transfer instrument includes the second carrier member, it is possible to transfer the medical sheet from the support portion to the treatment site by using the second carrier member without using another device (forceps or the like). It is therefore possible to efficiently transfer the medical sheet to the treatment site.

According to this transfer instrument, since the liquid can be supplied from the inside of the first shaft toward the support surface of the support portion through the liquid supply portion, the support surface of the support portion can be moistened with the liquid. As a result, when the distal portion of the second carrier member is slid on the unfolded support portion, the sliding of the distal portion of the second carrier member on the support portion can be improved. By supplying the liquid to the support portion, it is possible to suppress drying of the medical sheet unfolded outside the outer cylinder. Therefore, the medical sheet can be efficiently transferred to the treatment site without breakage.

(2) In the transfer instrument according to (1) above, the liquid supply portion may include: a liquid flow path formed inside the second shaft and extending in an axial direction of the second shaft; and a liquid supply hole that communicates with the liquid flow path and opens inside the first shaft to an outer peripheral surface of the second shaft.

According to this configuration, by supplying the liquid to the liquid flow path of the second shaft, the liquid is supplied between the first shaft and the second shaft via the liquid supply hole, so that the liquid can be effectively supplied to the support portion.

(3) In the transfer instrument according to (1) above, the liquid supply portion may include a liquid feeding tube disposed between an outer peripheral surface of the second shaft and the first shaft, and the liquid feeding tube may include a liquid outflow port that opens in the first shaft.

According to this configuration, by supplying the liquid to the liquid feeding tube, the liquid is supplied between the first shaft and the second shaft via the liquid outflow port, so that the liquid can be effectively supplied to the support portion.

(4) In the transfer instrument according to (3) above, the liquid feeding tube may be fixed to an outer peripheral surface of the second shaft, the first shaft may include a fitting portion to which the liquid feeding tube is fitted, and the fitting portion may regulate displacement of the liquid feeding tube with respect to the first shaft in a circumferential direction of the first shaft.

According to this configuration, since the liquid feeding tube and the fitting portion are fitted to each other, the second shaft is prevented from rotating relative to the first shaft in a lumen of the first shaft.

(5) In the transfer instrument according to (1) above, the first carrier member may include an injection port that is provided to a portion of the first shaft protruding in a proximal end direction from a proximal end of the outer cylinder and communicates with an inside of the first shaft.

According to this configuration, by providing the injection port, a liquid for priming can be supplied to the support portion with a simple configuration.

(6) In the transfer instrument according to any one of (1) to (5) above, the support portion may be formed of a flexible sheet having a front surface including the support surface and a back surface that is a surface opposite to the front surface, the support portion may include a pair of protrusions protruding upward from both sides in a width direction of the support surface orthogonal to a moving direction of the first shaft, and in a state where the support portion is retracted in the outer cylinder, the support portion may be retracted into the outer cylinder in a state of being curved and deformed such that both sides in a width direction of the support portion on the back surface are in contact with each other.

According to this configuration, when the support portion is retracted into the outer cylinder, the back surfaces of the protrusions formed by the pair of protrusions being curved and deformed to have a convex shape are in contact with each other, the pair of protrusions are displaced toward the support surface, and the support portion is curved and deformed into a heart shape. As a result, since the support portion can be retracted in a heart shape in the outer cylinder, it is possible to suppress breakage of the medical sheet held by the support portion as compared with a case where the support portion is deformed into a shape other than the heart shape. In addition, since the support portion can be smoothly and compactly retracted into the outer cylinder, a diameter of the outer cylinder can be reduced as compared with a configuration in which the support portion is not curved and deformed into a heart shape.

(7) In the transfer instrument according to (6) above, the liquid may be supplied to the liquid supply portion in a state where the support portion is retracted in the outer cylinder.

According to this configuration, in a state before the support portion is exposed in the distal end direction of the outer cylinder, it is possible to supply the liquid into the first shaft to maintain the retracted medical sheet in a wet state.

According to the present disclosure, by providing the liquid supply portion capable of supplying the liquid toward the support surface of the support portion through the distal-end opening of the first shaft, the liquid can be supplied from the inside of the first shaft toward the support surface of the support portion through the liquid supply portion, so that the support surface of the support portion can be moistened with the liquid. As a result, when the distal portion of the second carrier member is slid on the unfolded support portion, the sliding of the distal portion of the second carrier member on the support portion can be improved. By supplying the liquid to the support portion, it is possible to suppress drying of the medical sheet unfolded outside the outer cylinder. Therefore, the medical sheet can be efficiently transferred to the treatment site without breakage.

(8) A transfer instrument for transferring a medical sheet to a treatment site of a living body, the transfer instrument comprising: an outer cylinder; a first carrier member including a first shaft, the first shaft extending in an axial direction of the outer cylinder, and is disposed inside the outer cylinder to be movable along the axial direction, and a support portion disposed at a distal portion of the first shaft, and includes a support surface configured to hold the medical sheet when the distal portion of the first shaft is disposed in a distal end direction with respect to a distal portion of the outer cylinder; a second carrier member including a second shaft inserted through the first shaft, the second shaft extending along the first shaft and provided movably along the first shaft; and a liquid supply portion configured to supply a liquid into the first shaft in order to supply the liquid toward the support surface through a distal-end opening of the first shaft.

(9) A method of transferring a medical sheet to a treatment site of a living body, the method comprising: placing a transfer instrument for transferring the medical sheet to the treatment site of the living body in an initial state, the transfer instrument including an outer cylinder, a first carrier member including a first shaft, the first shaft extending in an axial direction of the outer cylinder, and is disposed inside the outer cylinder to be movable along the axial direction, and a first support portion disposed at a distal portion of the first shaft, a second carrier member including a second shaft inserted through the first shaft, the second shaft extending along the first shaft and provided movably along the first shaft and includes a second support portion disposed at a distal portion of the second shaft, and a liquid supply portion configured to supply a liquid into the first shaft in order to supply the liquid toward the first support surfaces through a distal-end opening of the first shaft, and wherein in the initial state, the first and second shafts are moved in a distal end direction relative to the outer cylinder so as to be at a protruding position where the first support portion and the second support portion protrude from the distal-end opening of the outer cylinder in the distal end direction; placing the medical sheet on the second support portion; retracting the medical sheet into the outer cylinder together with the first support portion and the second support portion from the initial position to a retracted position; inserting the transfer instrument inserted into the living body and positioning a distal end of the transfer instrument near the treatment site of the living body; moving the first shaft and the second shaft in a distal end direction relative to the outer cylinder, and unfolding the first support portion, the second support portion and the medical sheet; and moving the second carrier member in the distal end direction relative to the first carrier member so that the second support portion on which the medical sheet is placed is moved from the retracted position to an advanced position, and wherein the second support portion protrudes in the distal end direction relative to the distal end of the first support portion to move the medical sheet to above the treatment site of the living body and bringing an overhanging portion of the medical sheet into contact with the treatment site of the living body.

### BRIEF DESCRIPTION OF THE DRAWINGS

FIG. 1 is a perspective view of a transfer instrument according to a first embodiment of the present disclosure.

FIG. 2 is an exploded perspective view of the transfer instrument illustrated in FIG. 1.

FIG. 3 is a plan view of a distal portion of the transfer instrument illustrated in FIG. 1.

FIG. 4 is a longitudinal cross-sectional view taken along line IV-IV in FIG. 3.

FIG. 5 is a transverse cross-sectional view taken along line V-V in FIG. 3.

FIG. 6 is an enlarged perspective view of the transfer instrument of FIG. 1.

FIG. 7 is a transverse cross-sectional view taken along line VII-VII of FIG. 3.

FIG. 8 is a flowchart illustrating a procedure of a transfer method for transferring a medical sheet using the transfer instrument illustrated in FIG. 1.

FIG. 9 is an explanatory diagram of a preparing process.

FIG. 10 is a first explanatory diagram of a sheet placing process.

FIG. 11 is a second explanatory diagram of the sheet placing process.

FIG. 12 is an explanatory diagram of a retracting process.

FIG. 13 is a transverse cross-sectional view taken along line XIII-XIII in FIG. 12.

FIG. 14 is a transverse cross-sectional view taken along line XIV-XIV in FIG. 12.

FIG. 15 is an explanatory diagram of a positioning process.

FIG. 16 is an explanatory diagram of an unfolding process.

FIG. 17 is an explanatory diagram of a moving process.

FIG. 18 is an explanatory diagram of a withdrawing process.

FIG. 19 is a perspective view of a transfer instrument according to a second embodiment of the present disclosure.

FIG. 20 is a perspective view of a transfer instrument according to a third embodiment of the present disclosure.

FIG. 21 is a cross-sectional view taken along line XXI-XXI of FIG. 20.

FIG. 22 is a cross-sectional view of a transfer instrument including a movement regulation portion according to a first modification.

FIG. 23A is a plan view of a distal portion of a transfer instrument illustrating a movement regulation portion according to a second modification, and FIG. 23B is a plan view illustrating a state where a first support portion of the transfer instrument in

FIG. 23A is retracted in an outer cylinder.

FIG. 24 is a plan view of a distal portion of a transfer instrument including a first support portion according to a third modification.

FIG. 25 is a cross-sectional view taken along line XXV-XXV of FIG. 24.

FIG. 26 is an enlarged perspective view of a distal portion of a transfer instrument including a pressing body according to a fourth modification.

FIG. 27 is an enlarged perspective view of a distal portion of a transfer instrument including a pressing body according to a fifth modification.

FIG. 28 is a cross-sectional view of a distal portion of the transfer instrument of FIG. 27.

### DETAILED DESCRIPTION

Set forth below with reference to the accompanying drawings is a detailed description of embodiments of a transfer instrument for transferring a medical sheet to a treatment site of a living body representing examples of the inventive transfer instrument for transferring the medical sheet to the treatment site of the living body.

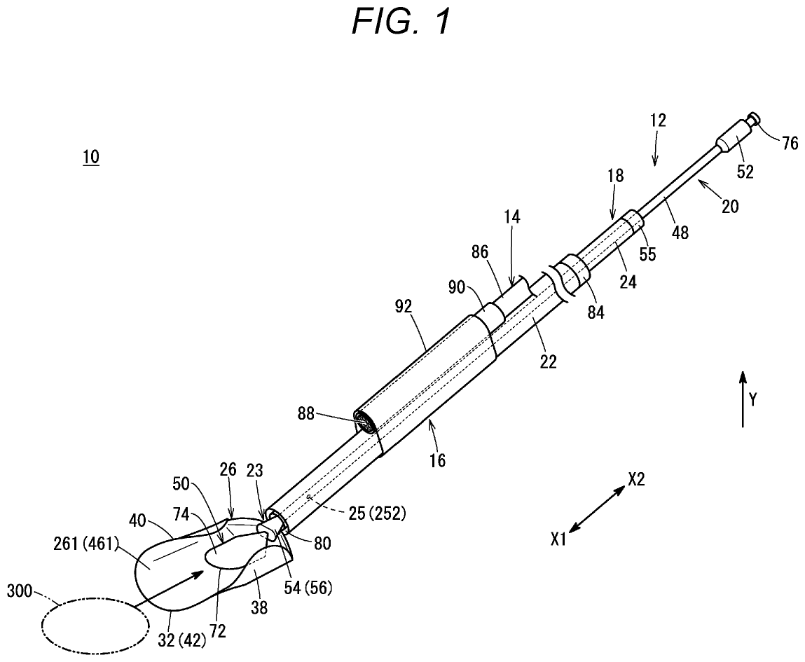

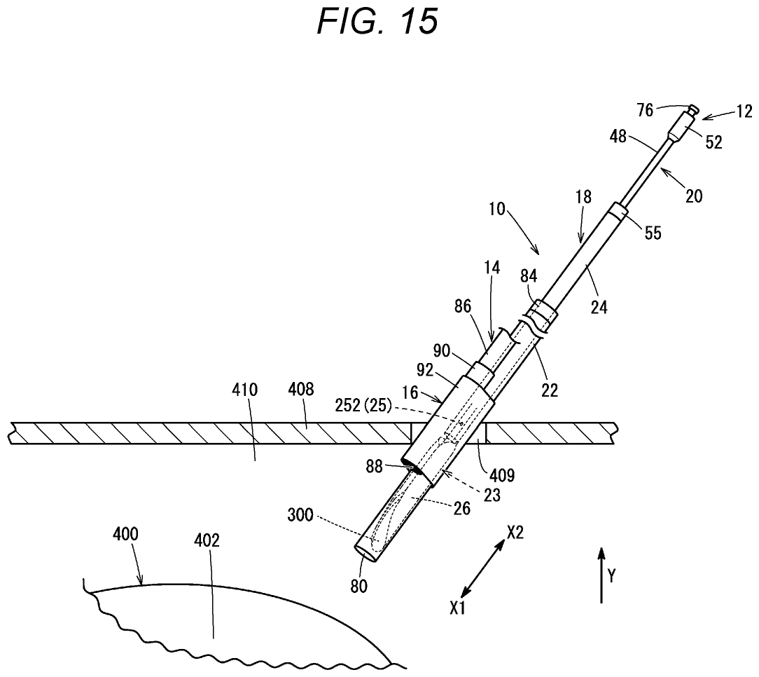

As illustrated in FIG. 1, a transfer instrument 10 according to the first embodiment is a medical instrument for transferring a medical sheet 300 to a treatment site of a living body. The transfer instrument 10 can be used for, for example, treatment of severe heart failure caused by ischemic heart disease. In this case, the medical sheet 300 is transplanted to a recipient site 402 of a heart 400 (the treatment site of the living body) (see FIGS. 15 to 18). The transfer instrument 10 is capable of attaching a plurality of the medical sheets 300 to the recipient site 402.

Examples of such a medical sheet 300 include pharmaceutical products or regenerative medicine products for medical use, a medical instrument, and the like. The medical sheet 300 is formed in a sheet shape such as a film shape or a membrane shape (gel object). Fibrin or the like may be applied to the medical sheet 300 for reinforcement. Examples of the regenerative medicine products including cells include a cell sheet (sheet-shaped cell culture), a spheroid, and the like. It is possible to form the cell sheet by culturing autologous cells or allogenic cells. The cells constituting the cell sheet can include, for example, somatic stem cells (adult stem cells), mesenchymal stem cells, or iPS cells (induced pluripotent stem cells)-derived cardiomyocytes. Examples of the somatic stem cells preferably include skeletal myoblast cells (myoblast cells).

The medical sheet 300 may contain a tissue adhesive, a local anesthetic, or the like. The medical sheet 300 has a thickness of, for example, about 100 μm, and has a diameter of, for example, about 60 mm. Note that the thickness and the diameter (size) of the medical sheet 300 can be set as desired.

The medical sheet 300 may be a sheet to be transplanted to an organ (for example, lung, liver, pancreas, kidney, small intestine, esophagus, or the like) other than the heart 400. Further, the medical sheet 300 may be, for example, an anti-adhesion sheet as long as the sheet is for medical use.

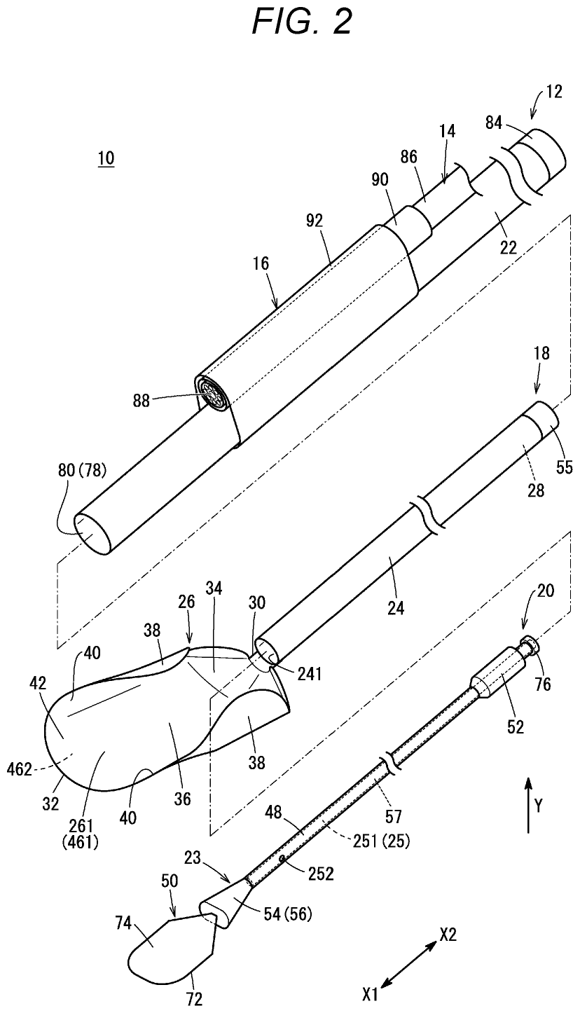

As illustrated in FIGS. 1 and 2, the transfer instrument 10 includes an instrument body 12, an endoscope 14, and a fixing member 16. An instrument body 12 includes a first carrier member 18, a second carrier member 20, an outer cylinder 22, a movement regulation portion 23, and a liquid supply portion 25. Note that the transfer instrument 10 is not limited to a configuration including the endoscope 14.

In FIG. 2, the first carrier member 18 includes a first shaft 24 and a first support portion 26.

The first shaft 24 is a tubular body (in the present embodiment, a circular tube member) including a first lumen 28. A distal end of the first shaft 24 includes a distal-end opening 241. The first lumen 28 opens at the distal end (end in the direction of arrow X1) of the first shaft 24 via the distal-end opening 241 and opens at a proximal end (end in the direction of arrow X2) of the first shaft 24. The first shaft 24 is provided with, at a proximal end of the first shaft 24, an airtight valve 55 that is in contact with an outer peripheral surface of a second shaft 48.

The first shaft 24 extends in an axial direction of the outer cylinder 22 and is disposed inside the outer cylinder 22 so as to be movable along the axial direction. The first shaft 24 can include, for example, a resin material. Examples of the constituent material of the first shaft 24 can include, but not particularly limited to, polyethylene, polypropylene, fluororesin, polyethylene terephthalate, polymethyl methacrylate, a polyamide resin, polystyrene, polycarbonate, polyimide, polyetherimide, polyetheretherketone, polyvinyl chloride, an ABS resin, a polyamide elastomer, and a polyester elastomer. The first shaft 24 may include a metal material.

The first shaft 24 may be flexible. The first shaft 24 may include a flexible tube portion capable of maintaining a bent shape. In this case, the first shaft 24 can be bent into any desired shape in a body cavity and is capable of maintaining the bent shape.

As illustrated in FIGS. 2 to 4, the first support portion 26 is attached to the distal portion of the first shaft 24. The first support portion 26 can include, for example, a resin material. The first support portion 26 can hold the medical sheet 300. A flexible resin sheet member (film member) is bent into a predetermined form to form the first support portion 26. Alternatively, the sheet member is shaped into a predetermined form by a sheet forming die to form the first support portion 26. It is preferable that the sheet member have, but not particularly limited to, a thickness of, for example, 100 um or more and 200 um or less. The first support portion 26 includes a first joint 30 and a first support body 32.

The constituent material of the first support portion 26 preferably has transparency, and examples of the constituent material of the first support portion 26 can include, but not particularly limited to, polyethylene, polycarbonate, polyamide, polystyrene, polypropylene, a polyacetal resin, polyimide, polyetherimide, polyetheretherketone, polyethylene terephthalate, and fluororesin. Further, the first support portion 26 may have a mesh shape.

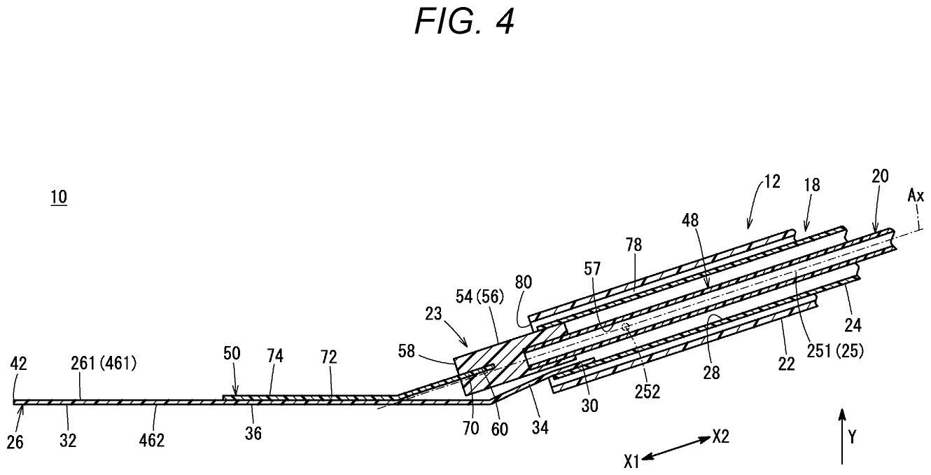

In FIG. 4, the first joint 30 is bonded to an inner peripheral surface of the distal portion of the first shaft 24 with an adhesive. Examples of the adhesive can include, but not particularly limited to, a UV adhesive, a hot-melt adhesive, and an instant adhesive (for example, cyanoacrylate-based instant adhesive). The first joint 30 may be thermally fused to the inner peripheral surface of the first shaft 24.

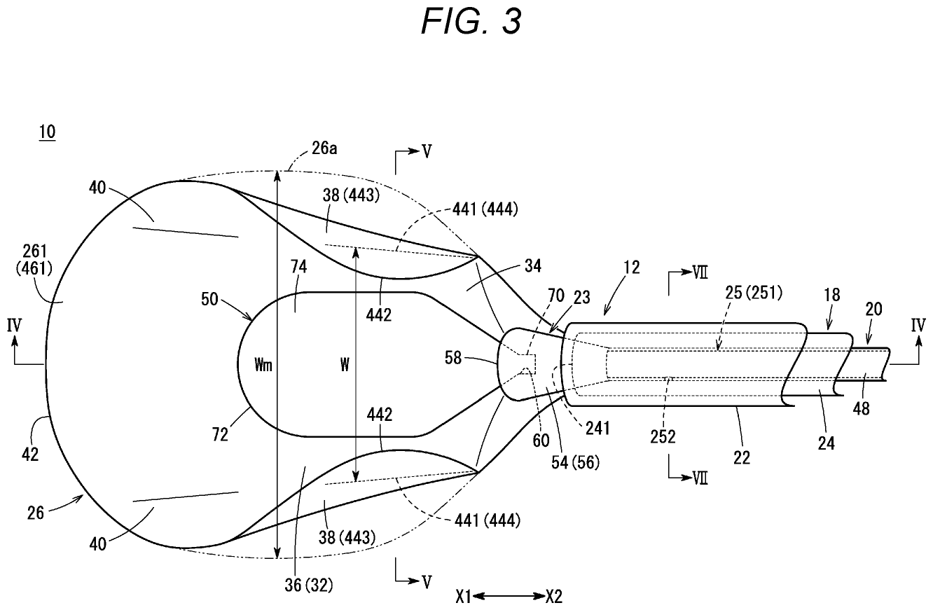

As illustrated in FIG. 3, the first support body 32 extends in a distal end direction from the first joint 30 (see FIG. 4). The first support body 32 includes a proximal-end support portion 34, an intermediate support portion 36, a pair of first protrusions 38, a pair of second protrusions 40, and a distal-end support portion 42.

The proximal-end support portion 34 extends roughly along an axis Ax of the first shaft 24 from a distal end of the first joint 30 in the distal end direction (the direction of arrow X1) (see FIG. 4). The proximal-end support portion 34 is formed to be wider in its extending direction. In other words, both sides of the proximal-end support portion 34 in a width direction are tapered toward the first joint 30.

The intermediate support portion 36 intersects with the axis Ax of the first shaft 24 in a distal end direction (in the direction of arrow X1), and extends from a distal end of the proximal-end support portion 34 in the distal end direction (in the direction of arrow X1) of the first support portion 26 (see FIG. 4). The intermediate support portion 36 is formed in a tapered shape so as to be gradually narrower in width from the distal end toward the proximal end (in the direction of arrow X2).

In FIGS. 2 and 3, the pair of first protrusions 38 protrude upward (in the direction of arrow Y) from both sides of the intermediate support portion 36 in the width direction orthogonal to a direction in which the first shaft 24 moves and inward in the width direction of the intermediate support portion 36. The pair of first protrusions 38 are connected to the proximal-end support portion 34.

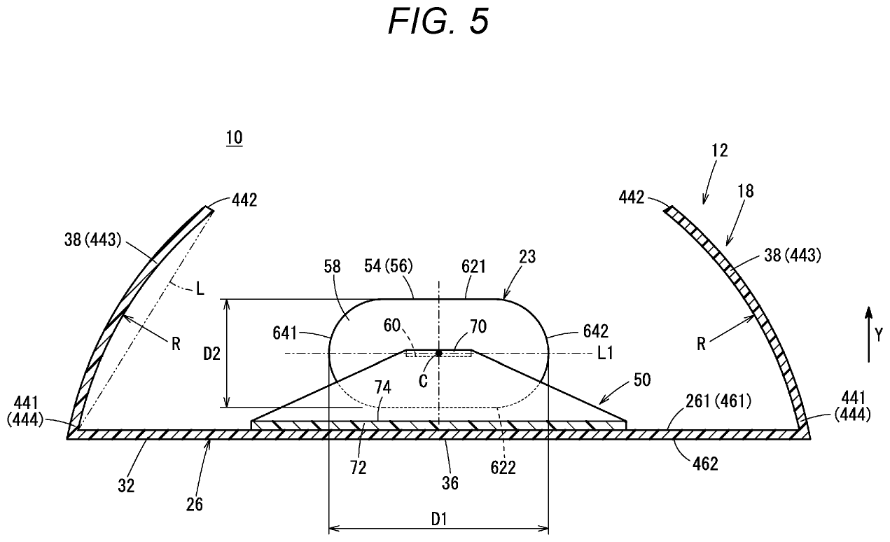

As illustrated in FIG. 5, each of the pair of first protrusions 38 includes a fixed end 441 connected to an upper surface (first support surface 261) of the intermediate support portion 36 and a free end 442 that is an end portion separated from the first support surface 261 in a protruding direction of the first protrusion 38.

In a cross section orthogonal to the protruding direction of the first protrusion 38 illustrated in FIG. 5 and orthogonal to the first support surface 261, each cross section of the first protrusions 38 includes an intermediate portion 443 constituting a portion between the free end 442 and the fixed end 441. The intermediate portion 443 has an arc shape bulging in a convex shape in a direction away from the first support surface 261. A cross section of the intermediate portion 443 is not limited to an arc shape having a single radius, and is only required to be an arc shape. In other words, in the cross section of the first protrusion 38 illustrated in FIG. 5, the intermediate portion 443 is disposed outward with respect to a line segment L connecting the fixed end 441 and the free end 442.

In a cross section orthogonal to the protruding direction of the first protrusion 38 illustrated in FIG. 5 and orthogonal to the first support surface 261, a curvature R of the intermediate portion 443 increases in a proximal end direction (the direction of arrow X2 in FIG. 3) of the first support portion 26.

As illustrated in FIG. 3, the pair of second protrusions 40 are connected to distal ends of the pair of first protrusions 38. The pair of second protrusions 40 protrude upward from both sides of the intermediate support portion 36 in the width direction and outward in the width direction of the intermediate support portion 36. Each second protrusion 40 is formed with a smaller curvature than the first protrusion 38. The second protrusions 40 are lower in protrusion height relative to the first support surface 261 than the first protrusions 38.

The distal-end support portion 42 is connected to a distal end of the intermediate support portion 36 and distal ends of the pair of second protrusions 40. The distal-end support portion 42 protrudes in an arc shape in the distal end direction (the direction of arrow X1). That is, when viewed from a direction orthogonal to the first support surface 261 illustrated in FIG. 3, the distal-end support portion 42 of the first support portion 26 has an arc shape connecting the pair of second protrusions 40.

Each of the pair of first protrusions 38 includes a pair of bent portions 444 on a proximal end side of the fixed end 441. Each of the pair of bent portions 444 causes a corresponding one of the pair of first protrusions 38 to bend from the first support surface 261 (intermediate support portion 36) of the first support portion 26 (see FIG. 5). As illustrated in FIG. 3, when the bent portion 444 of the first support portion 26 is bent to form the pair of first protrusions 38 bulging outward in a convex shape, it is preferable to bend both end portions at the bent portion 444 in a portion having a maximum width Wm in a width direction of a resin sheet member 26a (see an imaginary line shape in FIG. 3) that is to be the first support portion 26.

When viewed from a direction orthogonal to the first support surface 261 illustrated in FIG. 3, an interval W in a width direction of the pair of bent portions 444 in the first support portion 26 gradually decreases in the proximal end direction (the direction of arrow X2). That is, in the first support body 32, the pair of bent portions 444 are formed in a tapered shape in the proximal end direction.

The first support body 32 includes a front surface 461 that faces upward (in the direction of arrow Y) and includes the first support surface 261, and a back surface 462 that is a surface opposite to the front surface 461. The first support surface 261 includes a flat surface continuous over an upper surface of the proximal-end support portion 34 and upper surfaces of the intermediate support portion 36 and the distal-end support portion 42. A lubricant may be applied to the first support surface 261 so as to allow a second support portion 50 (to be described later) of the second carrier member 20 to smoothly slide on the first support surface 261.

As illustrated in FIG. 2, the second carrier member 20 includes a second shaft 48, the second support portion 50, and a hub 52.

The second shaft 48 is a tubular body (in the present embodiment, a circular tube member) including a second lumen 57. The second shaft 48 is longer in the axial direction than the first shaft 24. The second shaft 48 is inserted through the first lumen 28 of the first shaft 24 (see FIGS. 1 and 4). In other words, a distal portion of the second shaft 48 protrudes in the distal end direction (the direction of arrow X1) from the distal-end opening 241 of the first shaft 24. A proximal portion of the second shaft 48 protrudes in the proximal end direction (the direction of arrow X2) from the proximal-end opening of the first shaft 24 (see FIG. 1). The second shaft 48 is provided extending along the first shaft 24 so as to be movable along the first shaft 24.

Note that a resilient member such as a spring may be provided between the first lumen 28 of the first shaft 24 and the second shaft 48, and the second shaft 48 may be biased in the proximal end direction (the direction of arrow X2) with respect to the first shaft 24 by a resilient force of the resilient member. As a result, when the user moves the second shaft 48 in the distal end direction (the direction of arrow X1) with respect to the first shaft 24, the user's operation force disappears, so that the second shaft 48 is movable in the proximal end direction by the resilient force.

The second shaft 48 is configured to follow the shape of the first support portion 26. As the constituent material of the second shaft 48, for example, a material more flexible than the constituent material of the first shaft 24 is selected. Specifically, examples of the constituent material of the second shaft 48 include a polyamide elastomer, a polyester elastomer, a polyurethane elastomer, polyvinyl chloride, polybutadiene, a silicone rubber, and a metal coil (including a composite with a resin). The second shaft 48 is flexible.

As illustrated in FIG. 4, the second shaft 48 includes a carrier holding portion 54 and a pressure-application portion 56 that is the distal portion of the second shaft 48. The pressure-application portion 56 includes an elastic body such as an elastomer member. The pressure-application portion 56 presses the first support portion 26 against an inner surface of the outer cylinder 22 with the first support portion 26 retracted in the outer cylinder 22.

A distal end of the carrier holding portion 54 includes a pressing surface 58. The carrier holding portion 54 can cause the pressing surface 58 to press an outer edge surface of the medical sheet 300 supported by the first support portion 26 in the distal end direction (the direction of arrow X1). In the present embodiment, the pressure-application portion 56 is provided with the carrier holding portion 54 that supports the second support portion 50. The carrier holding portion 54 includes the pressing surface 58 and an attachment hole 60. The carrier holding portion 54 is formed to be wider in the distal end direction when viewed from above. The carrier holding portion 54 is formed in a trapezoidal shape when viewed from above. In a cross section orthogonal to an axis of the second shaft 48 illustrated in FIG. 5, the carrier holding portion 54 has a line-symmetrical shape with respect to an imaginary line L1 passing through a center line C of the carrier holding portion 54 and parallel to a width direction of the carrier holding portion 54. In other words, the carrier holding portion 54 has a symmetrical shape in a vertical direction with respect to the imaginary line L1.

Specifically, in a cross section orthogonal to the axis of the second shaft 48, a cross section of the carrier holding portion 54 includes a pair of linear portions 621, 622 orthogonal to the axis of the second shaft 48 and a pair of convex-shaped portions 641, 642 that are disposed on both sides of the pair of linear portions 621, 622 and are convex in a direction (outward in the width direction) away from the linear portions 621, 622. The convex-shaped portions 641, 642 are formed to have an arc cross-sectional shape. A first length D1 (width dimension) of the carrier holding portion 54 along an extending direction of the linear portions 621, 622 is larger than a second length D2 (thickness dimension) of the carrier holding portion 54 orthogonal to the extending direction. That is, the carrier holding portion 54 has a flattened cross-sectional shape. Note that the carrier holding portion 54 is not limited to a configuration formed in a flattened cross-sectional shape, and may be formed in, for example, a circular cross-sectional shape, an elliptical cross-sectional shape, a square cross-sectional shape, a rectangular cross-sectional shape, or the like. In addition, instead of the convex-shaped portions 641, 642, the carrier holding portion 54 may have an uneven structure in which the first support portion 26 is pressed against the inner surface of the outer cylinder 22 in a state where the first support portion 26 is retracted in the outer cylinder 22.

When the first support portion 26 is retracted in a lumen 78 of the outer cylinder 22 together with the pressure-application portion 56, the width (first length D1) of the pressure-application portion 56 is set such that the first support portion 26 is pushed toward the lumen 78 of the outer cylinder 22 by both end portions (pair of convex-shaped portions 641 and 642) of the pressure-application portion 56 in the width direction. The first length D1 of the pressure-application portion 56 is set to be equal to or slightly larger than an inner diameter of the outer cylinder 22 (a diameter of the lumen 78), for example. Since the first support portion 26 has a thickness, the first length D1 of the pressure-application portion 56 may be set slightly smaller than the inner diameter of the outer cylinder 22.

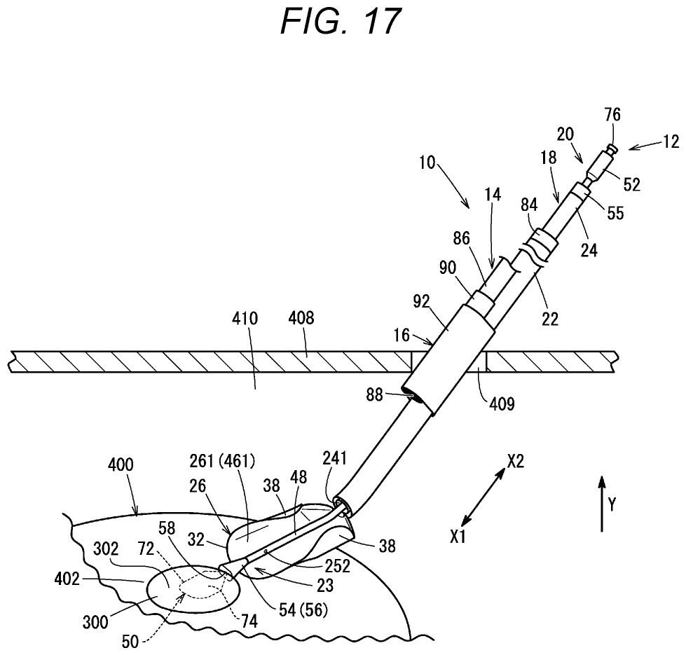

In FIG. 4, the pressing surface 58 is provided on a distal end surface of the carrier holding portion 54. The attachment hole 60 opens at the pressing surface 58. The second support portion 50 is attached to the pressing surface 58. The pressing surface 58 presses the outer edge surface of the medical sheet 300 in the distal end direction (the direction of arrow X1) (see FIG. 17). The pressing surface 58 is a flat surface, which is orthogonal to an axis of the carrier holding portion 54.

The attachment hole 60 opens in the pressing surface 58 of the carrier holding portion 54. The attachment hole 60 is disposed on the center line C of the carrier holding portion 54 on the pressing surface 58. The attachment hole 60 has a slit shape extending parallel to the width direction of the carrier holding portion 54 from the center line C. The attachment hole 60 has a symmetrical shape in the width direction with respect to the center line C. The attachment hole 60 extends in the axial direction from the pressing surface 58. A part of the second support portion 50 is inserted into and connected to the attachment hole 60.

In FIGS. 2 to 4, the second support portion 50 is a flexible sheet. The second support portion 50 includes a second joint 70 and a second support body 72. The second joint 70 is provided at a proximal end of the second support portion 50. The second joint 70 extends in a proximal end direction (the direction of arrow X2) from a proximal end of the second support body 72. The second joint 70 is inserted into and, for example, bonded to the attachment hole 60 of the carrier holding portion 54. The second joint 70 may be joined to the attachment hole 60 of the carrier holding portion 54 by a suitable joining method other than adhesion. Further, the second support portion 50 may be integrally formed with the carrier holding portion 54.

The second support body 72 extends in the distal end direction (the direction of arrow X1) from the second joint 70. The second support body 72 extending from the second joint 70 is shorter in the extending direction than the first support body 32 extending from the first joint 30. The second support body 72 is provided with, on its upper surface, a second support surface 74 on which the medical sheet 300 is placed. The second support surface 74 includes a flat surface. The second support body 72 is smaller than the first support body 32. That is, the second support surface 74 is smaller in area than the first support surface 261.

As illustrated in FIG. 3, a proximal portion of the second support body 72 is formed to be narrower in the proximal end direction (the direction of arrow X2). The intermediate portion between the distal end and the proximal end of the second support body 72 extends with a substantially constant width. The distal portion of the second support portion 50 protrudes in an arc shape toward the distal end direction (the direction of arrow X1). A lubricant may be applied to both surfaces (lower surface and upper surface) of the second support body 72.

In FIG. 2, the hub 52 is attached to the proximal portion of the second shaft 48. The hub 52 includes a connection port 76 to which a liquid supply instrument is detachably attached. The liquid supply instrument can supply a liquid (for example, a saline solution). The liquid can be, for example, for priming of a supply hole or for wetting of the first and/or second support surfaces.

In FIGS. 1 and 2, the outer cylinder 22 is a cylindrical member including the lumen 78. The lumen 78 includes a distal-end opening 80 that opens at a distal end (end in the direction of arrow X1) of the outer cylinder 22. The lumen 78 opens at a proximal end (end in the direction of arrow X2) of the outer cylinder 22. The outer cylinder 22 is flexible. Examples of the constituent material of the outer cylinder 22 are the same as the examples of the constituent material of the first shaft 24 described above. Note that, in a case where the first shaft 24 includes the flexible tube portion, the outer cylinder 22 can bend the first shaft 24. Further, in the present embodiment, the outer cylinder 22 may include the flexible tube portion as described above.

The first shaft 24 is inserted through the lumen 78 of the outer cylinder 22. The outer cylinder 22 is shorter in the axial direction than the first shaft 24. In FIGS. 3 and 4, an inner diameter of the outer cylinder 22 is smaller than a width of the intermediate support portion 36. The width of the intermediate support portion 36 is substantially identical to a circumferential length of an inner surface of the outer cylinder 22 or less than the circumferential length of the inner surface of the outer cylinder 22 so as to allow the first support portion 26 to be retracted into the outer cylinder 22 with the first support portion 26 curled into a cylinder along a circumferential direction of an inner peripheral surface of the outer cylinder 22. The outer cylinder 22 is provided with, at a proximal end of outer cylinder 22, an airtight valve 84 that is in contact or relatively close contact with an outer peripheral surface of the first shaft 24.

In FIGS. 2 and 4, a distal end surface of the outer cylinder 22 extends orthogonally to the axial direction of the outer cylinder 22.

As illustrated in FIG. 2, the endoscope 14 includes an elongated endoscope main body 86. A distal portion of the endoscope main body 86 is fixed to an outer peripheral surface of the outer cylinder 22 by the fixing member 16 (see FIG. 1). An objective lens 88 provided on a distal end surface of the endoscope main body 86 and the objective lens 88 is oriented such that the objective lens faces the distal end direction of the outer cylinder 22 (in the direction of arrow X1). The distal portion of the endoscope main body 86 is fixed to an intermediate portion of the outer cylinder 22 in the axial direction. Note that the distal portion of the endoscope main body 86 may be fixed to a distal portion of the outer cylinder 22.

The fixing member 16 can include, for example, a fixing cylinder 90 and a fixing tube 92. The fixing cylinder 90 can include, for example, a hard resin material. The endoscope main body 86 can be inserted into a lumen of the fixing cylinder 90. The fixing cylinder 90 is disposed along a longitudinal direction of the outer cylinder 22. The fixing tube 92 is a tube for fixing the fixing cylinder 90 at a predetermined position of the outer cylinder 22. The fixing tube 92 can be, for example, a heat-shrink tube. Note that the outer cylinder 22 and the fixing cylinder 90 may be integrally molded. Note that how to fix the distal portion of the endoscope main body 86 to the outer cylinder 22 may be determined as desired.

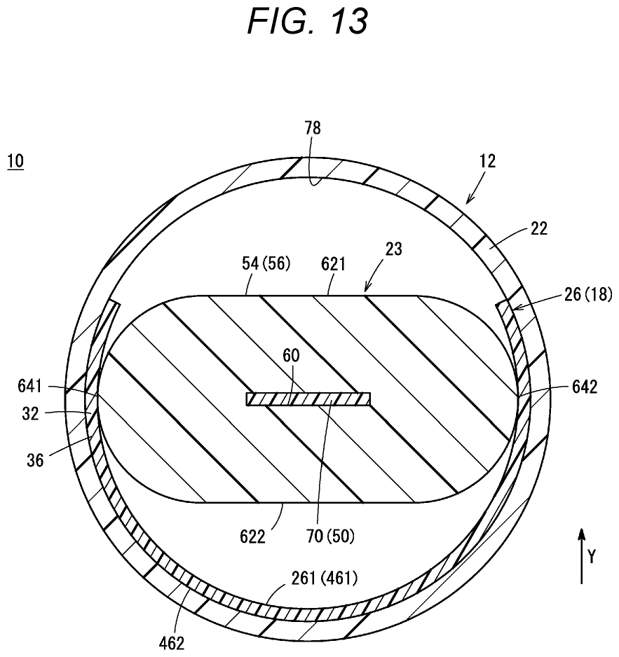

The movement regulation portion 23 can restrict relative movement between the first shaft 24 and the second shaft 48 in the axial direction. The movement regulation portion 23 is configured by the carrier holding portion 54 (pressure-application portion 56) configured by a distal portion of the second shaft 48. By the carrier holding portion 54 pressing the first support portion 26 against the inner surface (lumen 78) of the outer cylinder 22 in a state where the first support portion 26 is retracted in the outer cylinder 22, relative displacement between the first support portion 26 and the second shaft 48 is restricted. As the relative movement between the first support portion 26 and the second shaft 48 is restricted, relative movement between the first shaft 24 and the second shaft 48 in the axial direction is restricted (see FIG. 13).

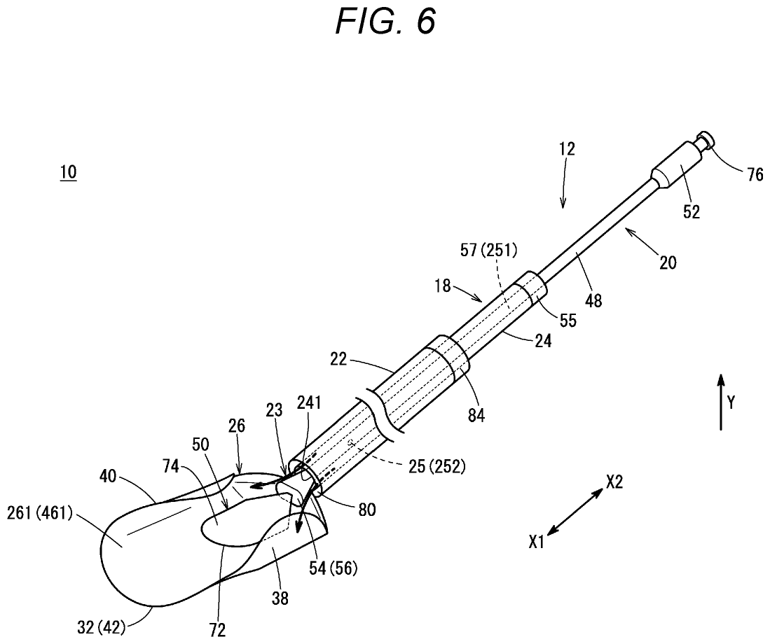

As illustrated in FIG. 6, the liquid supply portion 25 can supply a liquid for priming toward the first support portion 26 via the distal-end opening 241 of the first shaft 24. The liquid supply portion 25 includes a liquid flow path 251 formed inside the second shaft 48 and a liquid supply hole 252 that opens on the outer peripheral surface of the second shaft 48.

The liquid flow path 251 is the second lumen 57 of the second shaft 48. A proximal end of the liquid flow path 251 opens at a proximal end of the second shaft 48. The proximal end of the liquid flow path 251 communicates with the connection port 76 of the hub 52. The liquid flow path 251 communicates with the liquid supply hole 252. A liquid can flow through the liquid flow path 251 from the proximal end toward the distal end of the liquid flow path 251. Note that, the liquid flow path 251 may be provided separately from the second lumen 57 of the second shaft 48.

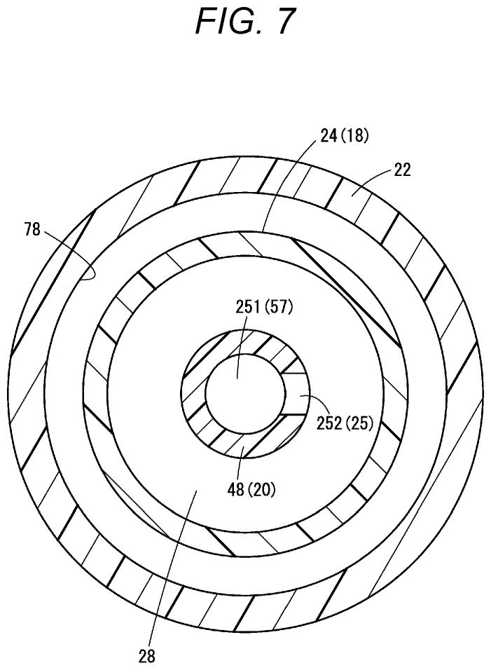

In the present embodiment, the liquid supply hole 252 is provided in the vicinity of the distal portion of the second shaft 48. The liquid supply hole 252 is disposed at a position separated from the pressure-application portion 56 of the second shaft 48 in the proximal end direction (the direction of arrow X2). The position of the liquid supply hole 252 is not limited to the vicinity of the distal portion of the second shaft 48. That is, when the second support portion 50 is located at a retracted position overlapping on the first support surface 261 of the first support portion 26 (when the second shaft 48 is moved in the proximal-most end direction relative to the first shaft 24), the liquid supply hole 252 may simply be located in the first lumen 28 of the first shaft 24. Therefore, the liquid supply hole 252 may be disposed not only in the vicinity of the distal portion of the second shaft 48 but also on the proximal end side of the second shaft 48 within a range of the first lumen 28 of the first shaft 24. As illustrated in FIG. 7, the liquid supply hole 252 penetrates an outer peripheral wall of the second shaft 48 in a radial direction, and the liquid supply hole 252 and the liquid flow path 251 communicate with each other. The liquid flow path 251 and the first lumen 28 of the first shaft 24 communicate with each other through the liquid supply hole 252.

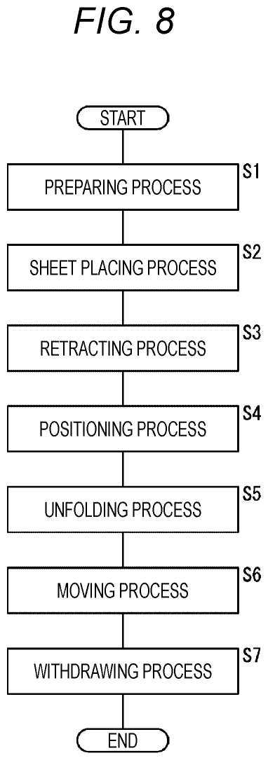

Next, a transfer method for transferring the medical sheet 300 to a treatment site of a living body will be described. Specifically, as illustrated in FIGS. 15 to 18, the transfer method for transferring the medical sheet 300 to the recipient site 402 of the heart 400 (the treatment site of the living body) during thoracoscopic surgery will be described. As illustrated in FIG. 8, the transfer method according to the present embodiment includes a preparing process, a sheet placing process, a retracting process, a positioning process, an unfolding process, a moving process, and a withdrawing process.

First, in the preparing process (S1), the transfer instrument 10 according to the present embodiment described above is prepared. The following description will be given on the assumption that a state as illustrated in FIG. 1 is an initial state of the transfer instrument 10. In the initial state, the first and second shafts 24 and 48 are moved in the distal end direction (the direction of arrow X1) relative to the outer cylinder 22 so as to be at a protruding position (second position) where the first support portion 26 and the second support portion 50 protrude from the distal-end opening 80 of the outer cylinder 22 in the distal end direction. The first and second support portions 26 and 50 are each unfolded when moving out of the outer cylinder 22 in the distal end direction, and the second support portion 50 is disposed on the first support surface 261 of the first support portion 26. That is, the second support portion 50 is disposed at a retracted position where the second support portion 50 is placed on the first support surface 261 of the first support portion 26. At this time, a proximal portion of the carrier holding portion 54 is in the first lumen 28 of the first shaft 24.

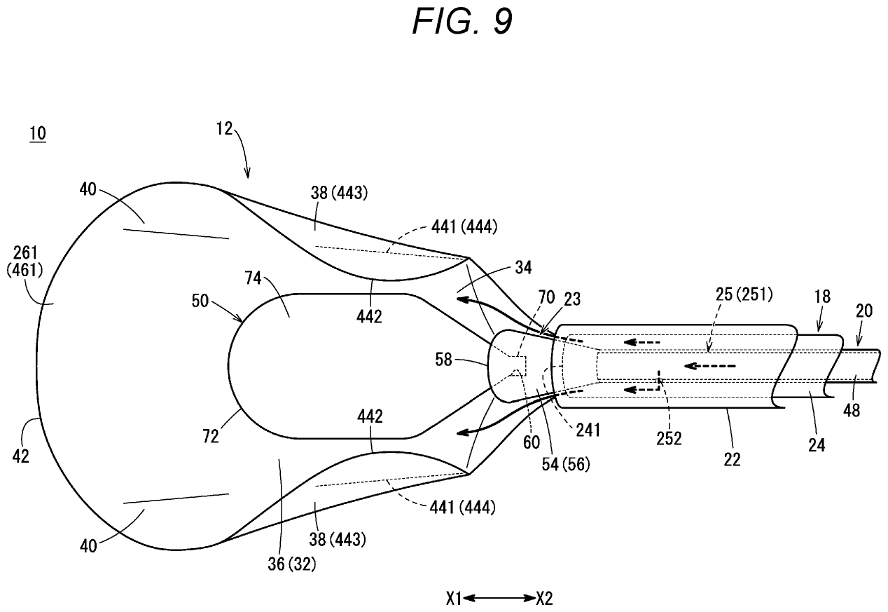

In the preparing process illustrated in FIG. 9, the user connects the liquid supply instrument to the hub 52 of the second carrier member 20, and operates the liquid supply instrument to supply a liquid to the liquid flow path 251. The liquid is discharged from the liquid supply hole 252 to radially outward of the second shaft 48 (the first lumen 28 of the first shaft 24), and flows toward the distal-end opening 241 of the first shaft 24 along an axial direction of the first and second shafts 24 and 48 through a space between an inner peripheral surface of the first shaft 24 and an outer peripheral surface of the second shaft 48. The liquid flowing out of the distal-end opening 241 is supplied to the first support surface 261 of the first support portion 26. As a result, the liquid is supplied between the first support surface 261 and the second support portion 50. Surface tension of the liquid causes the second support portion 50 to overlap with the first support surface 261 of the first support portion 26 in parallel.

After a predetermined amount of liquid is supplied to the first support surface 261 of the first support portion 26, the supply of the liquid to the liquid supply portion 25 is stopped by the user's operation on the liquid supply instrument. As a result, the supply of the liquid to the first support portion 26 by the liquid supply portion 25 is stopped.

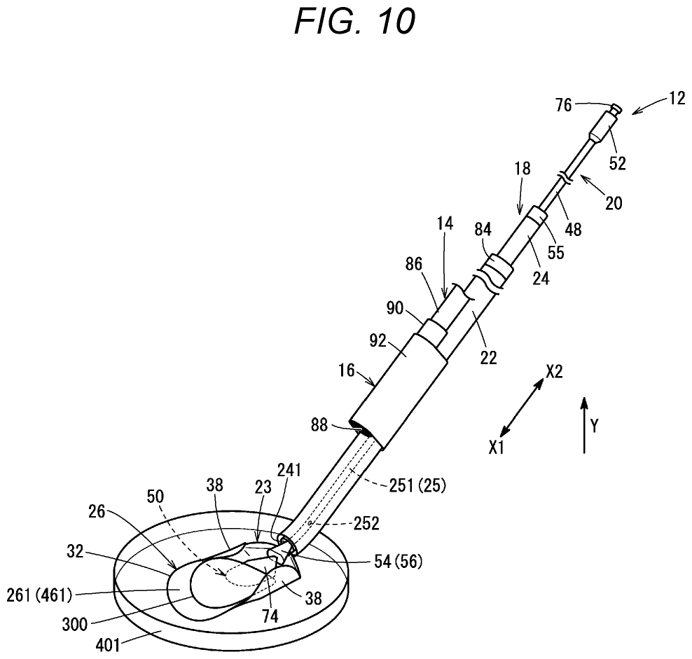

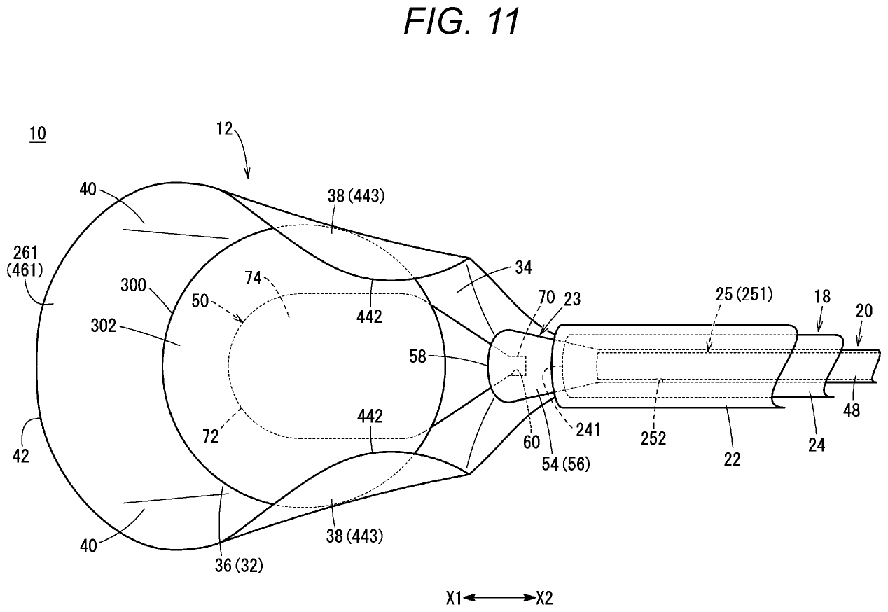

Next, in the sheet placing process (S2), as illustrated in FIG. 10, the medical sheet 300 placed on a Petri dish 401 is placed on the second support surface 74. Note that, as illustrated in FIG. 11, the medical sheet 300 extends out over the second support portion 50 with the medical sheet 300 placed on the second support surface 74. The first support surface 261 supports an overhanging portion 302 of the medical sheet 300 that extends out from the second support portion 50. The pair of second protrusions 40 help prevent the medical sheet 300 from moving (becoming misaligned) in the width direction of the intermediate support portion 36 with the medical sheet 300 placed on the second support surface 74.

Subsequently, in the retracting process (S3 in FIG. 8), the medical sheet 300 is retracted into the outer cylinder 22 together with the first support portion 26 and the second support portion 50 at a retracted position (first position). Specifically, the first shaft 24 of the first carrier member 18 and the second shaft 48 of the second carrier member 20 are moved together in the proximal end direction (the direction of arrow X2) relative to the outer cylinder 22.

Accordingly, the proximal-end support portion 34 is pulled in the proximal end direction through the distal-end opening 80 of the outer cylinder 22. At this time, when both the tapered sides of the proximal-end support portion 34 come into contact with the distal-end opening 80 of the outer cylinder 22, a force acts on the proximal-end support portion 34 to curl along the circumferential direction of the outer cylinder 22. Therefore, the proximal-end support portion 34 is smoothly pulled into the outer cylinder 22 while curling. At this time, the first support portion 26 is retracted into the outer cylinder 22 while curling in a conical shape such that the distal end side of the first support portion 26 becomes larger in diameter than the proximal-end support portion 34.

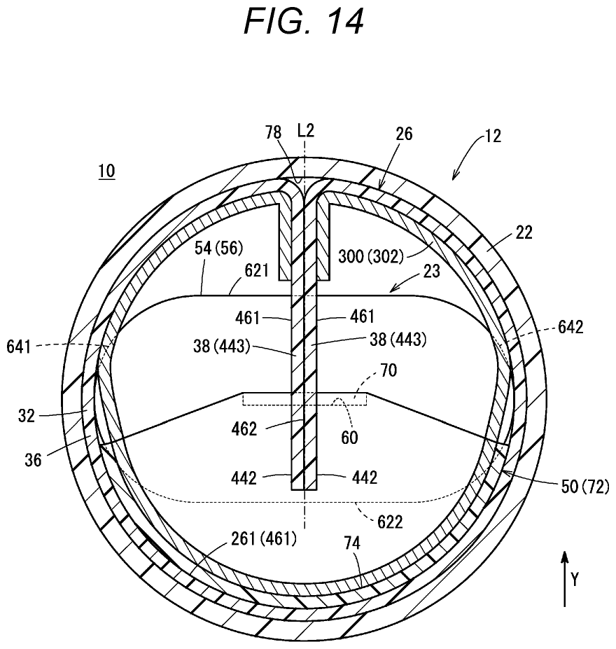

When the proximal-end support portion 34 becomes deformed, a force acts on the intermediate support portion 36 to cause the intermediate support portion 36 to curl along the circumferential direction of the outer cylinder 22, so that the intermediate support portion 36 is pulled into the outer cylinder 22 while curling. At this time, the intermediate support portion 36 becomes deformed into a cylindrical shape along the inner surface of the outer cylinder 22. Each of the pair of first protrusions 38 is curved such that its fixed end 441 is rolled inward, and the front surface 461 of the first support portion 26 faces inward, and the back surface 462 of the first support portion 26 faces outward. As illustrated in FIG. 14, the back surfaces 462 of the intermediate portions 443 bulging in a convex shape radially outward come into contact with each other on an imaginary line L2 extending in a direction orthogonal to a center axis of the outer cylinder 22. The intermediate portion 443 of one of the first protrusions 38 and the intermediate portion 443 of another first protrusion 38 come into contact with each other and are retracted downward (toward the first support surface 261, the front surface 461).

Accordingly, the back surface 462 of the first support portion 26 is curved to come into contact or relatively close contact with the inner surface of the outer cylinder 22, each of the first protrusions 38 is further curved from the fixed end 441 toward the free end 442 to fold back toward the center of the outer cylinder 22, and the pair of free ends 442 are disposed below the center axis of the outer cylinder 22. That is, the first support portion 26 is curved in a heart shape along the inner surface of the outer cylinder 22.

The heart shape refers to a roughly round shape including two convex shapes, i.e., a convex shape on one side and a convex shape on the other side. In a case where the heart shape is formed in the lumen 78 of the tubular body (outer cylinder 22), the two convex shapes protruding toward the opposite sides along the inner surface of the tubular body come close to each other so as to bring their respective peripheral surfaces into partial contact with each other, so that the entire contour becomes a roughly round shape along the inner surface of the tubular body (refer to the shape of the first support portion 26 in FIG. 14).

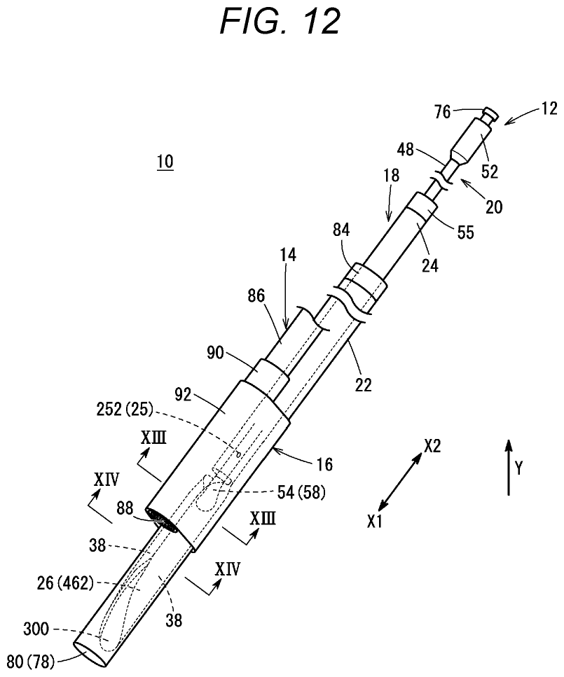

Along with the curved deformation of the first support portion 26, the second support portion 50 similarly undergoes curved deformation along the first support portion 26 inside the first support portion 26 (on the front surface 461 side). Along with the curved deformation of the first support portion 26 and the second support portion 50, the medical sheet 300 is deformed into a shape corresponding to the shape of the first support body 32 and the shape of the second support body 72, and the medical sheet 300 is retracted into the outer cylinder 22 accordingly. As illustrated in FIG. 12, when the first support portion 26 is entirely inserted into the outer cylinder 22, the retracting process is complete.

The liquid (for priming) may be supplied to the liquid flow path 251 in a state where the medical sheet 300 is retracted into the outer cylinder 22.

In the retracted state where the first support portion 26, the second support portion 50, and the medical sheet 300 are retracted into the outer cylinder 22, as illustrated in FIG. 14, the pair of first protrusions 38 are located adjacent to the distal end (in the direction of arrow X1) relative to the pressing surface 58 of the pressure-application portion 56 with the back surfaces 462 in contact with each other (see FIG. 12).

Subsequently, in the positioning process (S4 in FIG. 8), as illustrated in FIG. 15, the transfer instrument 10 is inserted into a chest cavity 410 through the incision 409 of the chest 408. At this time, the distal end of the transfer instrument 10 is positioned near the recipient site 402 of the heart 400, and the distal end of the endoscope 14 is positioned in the chest cavity 410. Note that before the transfer instrument 10 is inserted into the chest cavity 410, the liquid supply instrument may be connected to the connection port 76 of the hub 52 to introduce a liquid (for example, a saline solution).

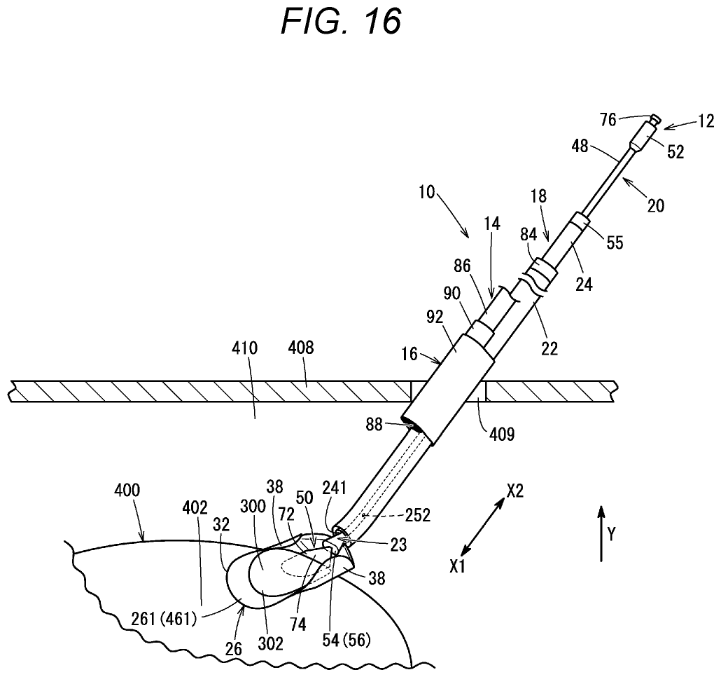

Subsequently, in the unfolding process (step S5 in FIG. 8), as illustrated in FIG. 16, the first support portion 26, the second support portion 50, and the medical sheet 300 are unfolded. Specifically, in the unfolding process, with the first shaft 24 gripped, the first shaft 24 is moved in the distal end direction (the direction of arrow X1) relative to the outer cylinder 22. Accordingly, the valve 55 of the first shaft 24 causes the second shaft 48 to move together with the first shaft 24 in the distal end direction (the direction of arrow X1). At this time, the first shaft 24 and the second shaft 48 integrally (i.e. together) move in the distal end direction via the movement regulation portion 23. Then, the first support portion 26 that has moved out of the distal-end opening 80 of the outer cylinder 22 returns to the original shape due to its restoring force. At the second position where the first support portion 26 is unfolded, the second support portion 50 and the medical sheet 300 unroll flat.

In the unfolding process, of the second carrier member 20, the second support surface 74 on which the medical sheet 300 is placed is entirely located on the first support surface 261. At this time, the medical sheet 300 is supported by the first support surface 261 and the second support surface 74. It is therefore possible to prevent the overhanging portion 302 of the medical sheet 300 from becoming crinkled before the medical sheet 300 is transferred to the recipient site 402 of the heart 400.

Next, in the moving process (S6 in FIG. 8), as illustrated in FIG. 17, the second carrier member 20 is moved in the distal end direction (the direction of arrow X1) relative to the first carrier member 18, so that the second support portion 50 on which the medical sheet 300 is placed is moved from the retracted position to an advanced position, and the second support portion 50 protrudes in the distal end direction (the direction of arrow X1) relative to the distal end of the first support portion 26. Specifically, in the moving process, the second shaft 48 is moved in the distal end direction relative to the first shaft 24.

Accordingly, the second support portion 50 moves in the distal end direction (the direction of arrow X1) relative to the first support portion 26. At this time, when the pressing surface 58 of the carrier holding portion 54 (pressure-application portion 56) presses the outer edge surface of the medical sheet 300 in the distal end direction, the medical sheet 300 is entirely located further than the first support portion 26 in the distal end direction. In this moving process, the medical sheet 300 is moved to above the recipient site 402 of the heart 400 to bring the overhanging portion 302 of the medical sheet 300 into contact with the recipient site 402.

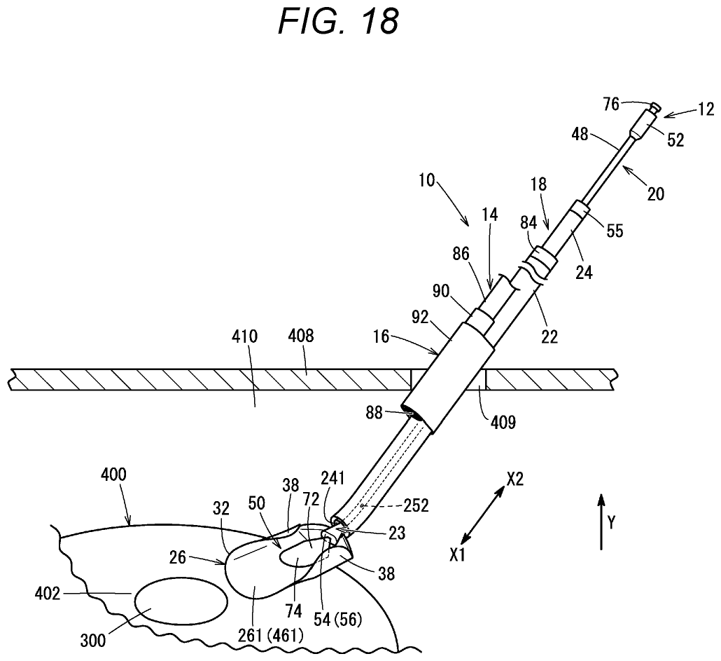

Subsequently, in the withdrawing process (S7 in FIG. 8), as illustrated in FIG. 18, the second carrier member 20 is moved from the second position to the first position to withdraw the second support portion 50 from between the recipient site 402 and the medical sheet 300. Accordingly, the medical sheet 300 entirely comes into contact with a front surface of the recipient site 402. This is the end of the transfer of the medical sheet 300 to the recipient site 402. Subsequently, the transfer instrument 10 is withdrawn from the chest 408 with the first support portion 26 and the second support portion 50 retracted into the outer cylinder 22.

The present embodiment has the following effects.

Since the transfer instrument 10 includes the second carrier member 20 movable in the axial direction relative to the first carrier member 18, it is possible to transfer the medical sheet 300 from above the first support portion 26 to the recipient site 402 of the living body using the second carrier member 20 without using another device (forceps or the like). It is therefore possible to efficiently transfer the medical sheet 300 to the recipient site 402.

As illustrated in FIG. 9, since the liquid can be supplied from the inside of the first shaft 24 toward the first support surface 261 of the first support portion 26 through the liquid supply portion 25, the first support surface 261 of the first support portion 26 can be moistened with the liquid. As a result, when the distal portion of the second carrier member 20 is slid on the first support surface 261 of the unfolded first support portion 26 at a protruding position where the first support portion 26 protrudes with respect to the outer cylinder 22, the sliding of the distal portion of the second carrier member 20 on the first support portion 26 can be improved. By supplying the liquid to the first support portion 26, it is possible to suppress drying of the medical sheet 300 unfolded outside the outer cylinder 22.

The liquid supply portion 25 includes the liquid flow path 251 formed inside the second shaft 48 and extending in an axial direction of the second shaft 48, and the liquid supply hole 252 that communicates with the liquid flow path 251 and opens inside the second shaft 48 to an outer peripheral surface of the first shaft 24. By supplying the liquid to the liquid flow path 251, the liquid is supplied between the first shaft 24 and the second shaft 48 via the liquid supply hole 252, so that the liquid can be effectively supplied from the distal-end opening 241 of the first shaft 24 to the first support portion 26.

When the liquid is supplied to the liquid supply portion 25 in a state where the first support portion 26 illustrated in FIG. 12 is retracted in the lumen 78 of the outer cylinder 22, the medical sheet 300 held by the first support portion 26 in the outer cylinder 22 can be maintained in a wet state with the liquid.

As illustrated in FIG. 2, the first support portion 26 is formed of a flexible sheet including the front surface 461 and the back surface 462. The first support portion 26 includes the pair of first protrusions 38 protruding upward from both sides of the first support surface 261 in the width direction orthogonal to the direction in which the first shaft 24 moves. With this configuration, as illustrated in FIG. 14, when the first support portion 26 is retracted into the outer cylinder 22, the back surfaces 462 of the pair of first protrusions 38 curved and deformed into a convex shape come into contact with each other. The pair of first protrusions 38 are displaced toward the first support surface 261, and the first support portion 26 is curved and deformed into a heart shape. Accordingly, the first support portion 26 can be retracted with the heart shape in the outer cylinder 22, so that it is possible to effectively reduce the possibility of breakage of the medical sheet 300 held by the first support portion 26 as compared with a case where the first support portion 26 is deformed into a shape other than the heart shape. Since the first support portion 26 can be smoothly and compactly retracted into the outer cylinder 22, the diameter of the outer cylinder 22 can be reduced as compared with a configuration where the first support portion 26 is not curved and deformed into a heart shape.

As illustrated in FIG. 13, in a state where the first support portion 26 is retracted in the outer cylinder 22, relative movement between the first shaft 24 and the second shaft 48 in the axial direction is prevented by the movement regulation portion 23 bringing the distal portion of the second shaft 48 and the first support portion 26 into contact with each other. As a result, even if the user erroneously attempts to move the second carrier member 20 in the distal end direction (the direction of arrow X1) before the second position is reached where the first support portion 26 protrudes from the distal portion of the outer cylinder 22, the second carrier member 20 does not move relative to the first carrier member 18 in the axial direction. Specifically, even if the user erroneously grips and pushes the second carrier member 20 in the distal end direction, the first carrier member 18 and the second carrier member 20 both do not move in the distal end direction with respect to the outer cylinder 22. However, there may be a case in which, when the user erroneously grips and pushes the second carrier member 20 in the distal end direction, the first carrier member 18 also moves in the distal end direction with respect to the outer cylinder 22 together with the second carrier member 20. Also in this case, the second carrier member 20 does not move relative to the first carrier member 18 in the axial direction. As a result, the second support portion 50 and the medical sheet 300 are prevented from protruding from the distal-end opening 80 of the outer cylinder 22. As a result, the medical sheet 300 retracted in the outer cylinder 22 is prevented from being pushed and broken by the distal portion of the second shaft 48. Therefore, the medical sheet 300 can be efficiently transferred to the recipient site 402 without breakage.

The movement regulation portion 23 is the pressure-application portion 56 constituting the distal portion of the second shaft 48. In a state where the first support portion 26 is retracted in the outer cylinder 22, the pressure-application portion 56 presses the first support portion 26 against the lumen 78 (inner surface) of the outer cylinder 22. As a result, the pressure-application portion 56 constituting the movement regulation portion 23 and the first support portion 26 are brought into contact or relatively close contact with each other in the radial direction, so that relative displacement between the first support portion 26 and the second shaft 48 is restricted. The second shaft 48 can be made movable in the axial direction with respect to the first shaft 24 in a state where the first support portion 26 protrudes from the distal portion of the outer cylinder 22.

Since the pressure-application portion 56 includes an elastic body, the pressure-application portion 56 can be effectively brought into contact or relatively close contact with the first support portion 26. Even if the second shaft 48 is pushed in the distal end direction (the direction of arrow X1) when the pressure-application portion 56 is retracted into the outer cylinder 22, the second shaft 48 is bent and a pressing force is less likely to be transmitted to the pressure-application portion 56. Therefore, the user easily notices an erroneous operation.

In a cross section orthogonal to the axis of the second shaft 48, the pressure-application portion 56 includes the pair of linear portions 621, 622 and the pair of convex-shaped portions 641, 642 provided on both sides of the pair of linear portions 621, 622. The first length D1 along the extending direction of the linear portions 621, 622 is longer than the second length D2 (see FIG. 5). As a result, when the first support portion 26 is retracted into the outer cylinder 22, when the first support portion 26 is pushed toward the outer cylinder 22 with the pair of convex-shaped portions 641, 642 provided on both sides of the linear portions 621, 622, the first support portion 26 and the carrier holding portion 54 can be brought into closer contact with each other.

As illustrated in FIG. 1, the second support portion 50 having a sheet shape and including the second support surface 74 capable of holding the medical sheet 300 is provided to the distal portion of the second shaft 48. The second support portion 50 is movable relative to the first support portion 26 between the retracted position where the second support portion 50 is placed on the first support surface 261 and the advanced position located adjacent to the distal end (in the direction of arrow X1) relative to the first support surface 261. With this configuration, the medical sheet 300 can be moved along the first support surface 261 of the first support portion 26 by the second support portion 50 including the second support surface 74.

As illustrated in FIG. 12, at the retracted position (first position), the second support portion 50 is retracted in the outer cylinder 22 together with the first support portion 26 with the second support portion 50 curved and deformed. With this configuration, even the second support portion 50 having a width dimension can be suitably retracted inside the outer cylinder 22 together with the first support portion 26 in the retracting process.

The carrier holding portion 54 that holds a proximal portion of the second support portion 50 is provided to the distal portion of the second shaft 48. Unlike the present embodiment, in a case where the carrier holding portion 54 does not have a vertically symmetrical shape and the second support portion 50 is held at a lower end of the carrier holding portion 54, the user rotates the second shaft 48 to reverse a vertical direction of the second support portion 50, and the second support portion 50 is disposed above the carrier holding portion 54. In this case, the second support portion 50 rises with respect to the first support surface 261 of the first support portion 26, and the first support portion 26 and the second support portion 50 are separated in the vertical direction.

On the other hand, in the present embodiment, as illustrated in FIG. 5, the carrier holding portion 54 is formed in line symmetry (vertical symmetry) with respect to the center line C of the carrier holding portion 54, in the cross section orthogonal to the axis of the second shaft 48. Further, the carrier holding portion 54 holds the second support portion 50 on the center line C. Therefore, even when the vertical direction of the second support portion 50 is reversed by the user rotating the second shaft 48, rising of the second support portion 50 from the first support surface 261 of the first support portion 26 can be suppressed as compared with the configuration in which the second support portion 50 is held at the lower end of the carrier holding portion 54. As a result, the medical sheet 300 can be stably subjected to a transplant procedure by the second support portion 50.

In a cross section orthogonal to the protruding direction of the first protrusion 38 and orthogonal to the first support surface 261, each cross section of the first protrusions 38 has a shape in which the intermediate portion 443 between the free end 442 and the fixed end 441 of the first protrusion 38 bulges in a convex shape in a direction away from the first support surface 261. With this configuration, when the first support portion 26 is retracted into the outer cylinder 22, the first support portion 26 can be reliably curved and deformed into a heart shape. As a result, it is possible to prevent breakage of the medical sheet 300 due to the deformation of the first support portion 26 into a shape other than the heart shape.

Unlike the present embodiment, in a case where the intermediate portion 443 has a curved shape so as to be concaved toward the first support surface 261 on the back surface 462 of the first support portion 26, the first support portion 26 may not be curved and deformed into an appropriate heart shape in the outer cylinder 22 in some rare cases. For example, since the free end 442 (front surface 461) of one first protrusion 38 rides on the fixed end 441 (back surface 462) of another first protrusion 38 that is curved and deformed, the free end 442 is bent in a V shape toward the inner surface of the outer cylinder 22 in some rare cases. In the present embodiment, since the intermediate portion 443 has a shape bulging in a convex shape in a direction away from the first support surface 261, it is possible to prevent the V-shaped bending of the first support portion 26.

By forming the intermediate portion 443 of the pair of first protrusions 38 in an arc shape, the first protrusion 38 having the shape bulging outward can be easily formed.

Since the curvature R of the intermediate portion 443 of the first protrusion 38 increases in the proximal end direction (the direction of arrow X2) of the first support portion 26, the interval W in the width direction between the first protrusions 38 on the distal end side of the first protrusion 38 can be widened. As a result, the medical sheet 300 can be easily placed on the first support surface 261 of the first support portion 26 through the distal-end support portion 42.

As illustrated in FIG. 3, the interval W between the pair of bent portions 444 in the width direction of the first support portion 26 decreases in the proximal end direction. Therefore, when the first support portion 26 is retracted into the outer cylinder 22 from the proximal end side, the pair of first protrusions 38 can be suitably curved and deformed in a conical shape by being in contact with the outer cylinder 22. Accordingly, interference between the proximal ends of the pair of bent portions 444 is suppressed, and thus the first support portion 26 can be smoothly retracted into the outer cylinder 22 from the proximal end side.

The distal portion of the first support portion 26 has an arc shape connecting the pair of second protrusions 40 when viewed from a direction orthogonal to the first support surface 261. Therefore, sliding resistance between the first support portion 26 and the outer cylinder 22 can be reduced when the first support portion 26 is retracted into the outer cylinder 22, as compared with a configuration in which the distal portion of the first support portion 26 has a shape having a corner portion. When the medical sheet 300 is placed on the first support portion 26 by moving the first support portion 26 in the distal end direction (the direction of arrow X1), the work can be smoothly performed by gradually inserting the first support portion 26 into the interface between the medical sheet 300 and the Petri dish 401 (container).

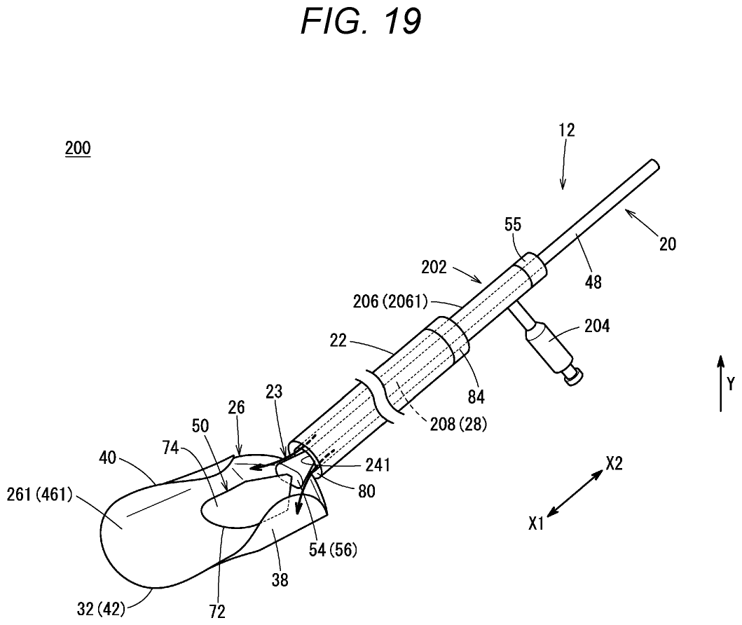

As illustrated in FIG. 19, a transfer instrument 200 of a second embodiment includes an injection port 204 provided to a first carrier member 202. The injection port 204 is provided to an exposed portion 2061 of a first shaft 206 protruding in a proximal end direction from a proximal end of an outer cylinder 22. The injection port 204 is formed in a tubular shape and protrudes from the first shaft 206. The injection port 204 communicates with a first lumen 28 of the first shaft 206. The first lumen 28 is a liquid flow path 208 through which a liquid can flow. The injection port 204 can be connected with a liquid supply instrument such as a syringe or a pump capable of supplying a liquid for priming. In the transfer instrument 200, a hub 52 (see FIG. 1) is not provided at a proximal portion of a second shaft 48. The second shaft 48 does not need to be a tubular body, and may have a solid structure. By connecting the liquid supply instrument to the injection port 204 and supplying the liquid from the liquid supply instrument to the injection port 204, the liquid is supplied from a distal-end opening 241 of the first shaft 206 to a first support surface 261 of a first support portion 26 through between the first shaft 206 and the second shaft 48. Note that, instead of providing the hub 52, a handle that can be gripped by the user may be provided at a proximal end of the second shaft 48.

As described above, the first carrier member 202 of the transfer instrument 200 according to the second embodiment includes the injection port 204 in the exposed portion 2061 protruding in the proximal end direction from the proximal end of the outer cylinder 22, and the injection port 204 communicates with the first lumen 28 (liquid flow path 208) of the first shaft 206. Thus, the liquid for priming can be supplied to the first support portion 26 with a relatively simple configuration in which the injection port 204 is provided to the first shaft 206.

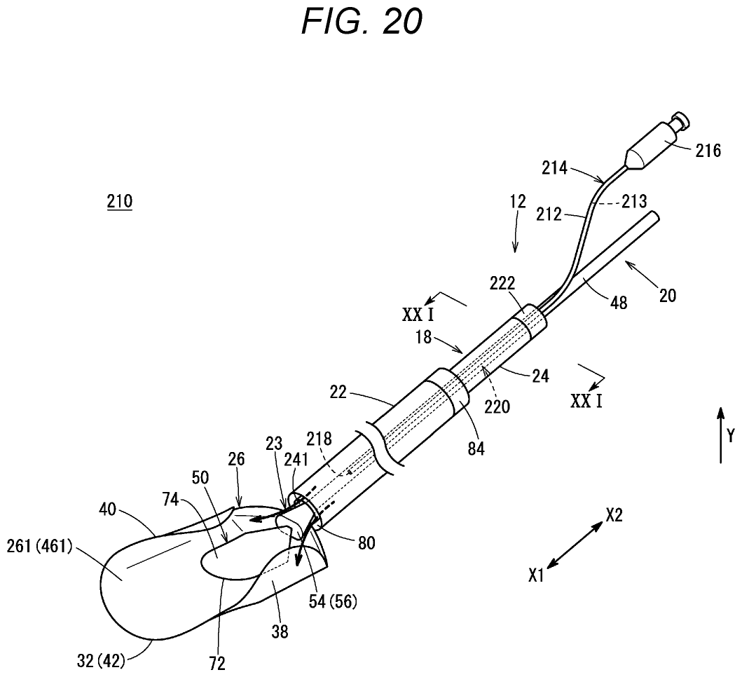

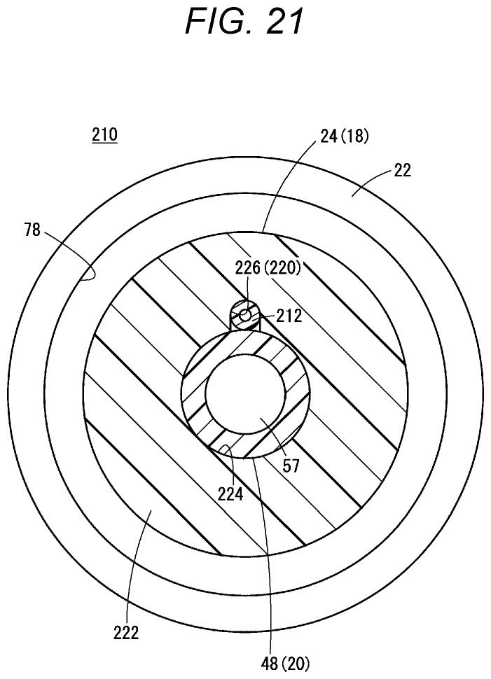

As illustrated in FIG. 20, a transfer instrument 210 according to a third embodiment includes a liquid supply portion 214 including a liquid feeding tube 212. In the transfer instrument 210, a hub 52 (see FIG. 1) is not provided at a proximal portion of a second shaft 48. The second shaft 48 does not need to be a tubular body, and may have a solid structure. A lumen of the liquid feeding tube 212 is a liquid flow path 213. The liquid feeding tube 212 is formed in, for example, a tubular shape having a circular cross-sectional shape. A part of the liquid feeding tube 212 is disposed inside a first shaft 24 (between an outer peripheral surface of the second shaft 48 and an inner peripheral surface of the first shaft 24). The liquid feeding tube 212 extends along an axial direction of the second shaft 48 and is fixed to the outer peripheral surface of the second shaft 48. In a cross section of the second shaft 48 orthogonal to the axial direction of the second shaft 48 illustrated in FIG. 21, the liquid feeding tube 212 protrudes radially outward from the outer peripheral surface of the second shaft 48. Note that, instead of providing the hub 52, a handle that can be gripped by the user may be provided at a proximal end of the second shaft 48.

As illustrated in FIG. 20, a proximal end of the liquid feeding tube 212 extends in a proximal end direction (the direction of arrow X2) relative to a proximal end of the first shaft 24. The proximal end side of the liquid feeding tube 212 is exposed outward from the proximal end of the first shaft 24. A hub 216 is fixed to a proximal portion of the liquid feeding tube 212. To the hub 216, a liquid supply instrument such as a syringe or a pump capable of supplying a liquid for priming is detachably attached. In the present embodiment, the proximal portion of the liquid feeding tube 212 is not fixed to the outer peripheral surface of the second shaft 48 but is a free end away from the second shaft 48. The proximal portion of the liquid feeding tube 212 may be fixed to the outer peripheral surface of the second shaft 48.

The distal end of the liquid feeding tube 212 includes a liquid outflow port 218 through which the liquid flows out. The liquid outflow port 218 opens at a distal end of the liquid feeding tube 212. The distal end of the liquid feeding tube 212 including the liquid outflow port 218 is disposed inside a first lumen 28 of the first shaft 24. The position where the liquid outflow port 218 is provided is not limited to the distal end of the liquid feeding tube 212, and may be on the proximal end side relative to the distal end of the liquid feeding tube 212. In a state where the second shaft 48 is displaced to the proximal-most end side relative to the first shaft 24, the liquid outflow port 218 is not exposed outward of the first shaft 24. The liquid outflow port 218 communicates with the liquid flow path 213.

The first shaft 24 includes a fitting portion 220 to which the liquid feeding tube 212 is fitted. The fitting portion 220 is provided to a valve 222 disposed at the proximal end of the first shaft 24. As illustrated in FIG. 21, the valve 222 includes a shaft hole 224 through which the second shaft 48 can be inserted, and includes the fitting portion 220 recessed radially outward from an inner peripheral surface of the shaft hole 224.

The fitting portion 220 includes a groove 226 recessed radially outward from an inner peripheral surface of the first shaft 24. The groove 226 extends along an axial direction of the valve 222. The groove 226 extends from a proximal end of the valve 222 to a distal end of the valve 222. The liquid feeding tube 212 can be inserted through the groove 226. When the liquid feeding tube 212 is inserted into the groove 226 inside the first shaft 24, relative movement of the liquid feeding tube 212 with respect to the first shaft 24 in a circumferential direction of the first shaft 24 is restricted. The liquid feeding tube 212 is movably fitted along the extending direction of the groove 226. Therefore, the first shaft 24 and the second shaft 48 are relatively movable in the axial direction.

In FIG. 20, when the liquid supply instrument) is connected to the hub 216 provided at the proximal end of the liquid feeding tube 212, and the liquid is supplied from the liquid supply instrument to the liquid feeding tube 212 (liquid flow path 213), the liquid is supplied in the distal end direction along the liquid feeding tube 212 between the first shaft 24 and the second shaft 48. After the liquid flows out between the first shaft 24 and the second shaft 48 from the liquid outflow port 218 of the liquid feeding tube 212, the liquid is supplied from the distal-end opening 241 of the first shaft 24 to the first support surface 261 of the first support portion 26.

As described above, in the transfer instrument 210 according to the third embodiment, the liquid supply portion 214 includes the liquid feeding tube 212 disposed between the outer peripheral surface of the second shaft 48 and the first shaft 24. The liquid feeding tube 212 includes the liquid outflow port 218 that opens in the first shaft 24. As a result, by supplying the liquid to the liquid feeding tube 212, the liquid is supplied between the first shaft 24 and the second shaft 48 via the liquid outflow port 218, and the liquid can be effectively supplied to the first support portion 26. In the first lumen 28 of the first shaft 24, when the fitting portion 220 and the liquid feeding tube 212 are fitted to each other, the second shaft 48 is prevented from rotating relative to the first shaft 24.

In a state where a second support surface 74 of a second support portion 50 is disposed facing upward, the liquid feeding tube 212 is disposed above the outer peripheral surface of the second shaft 48 in the second shaft 48, so that the liquid feeding tube 212 can be used as a marker for confirming that the second support surface 74 faces upward.

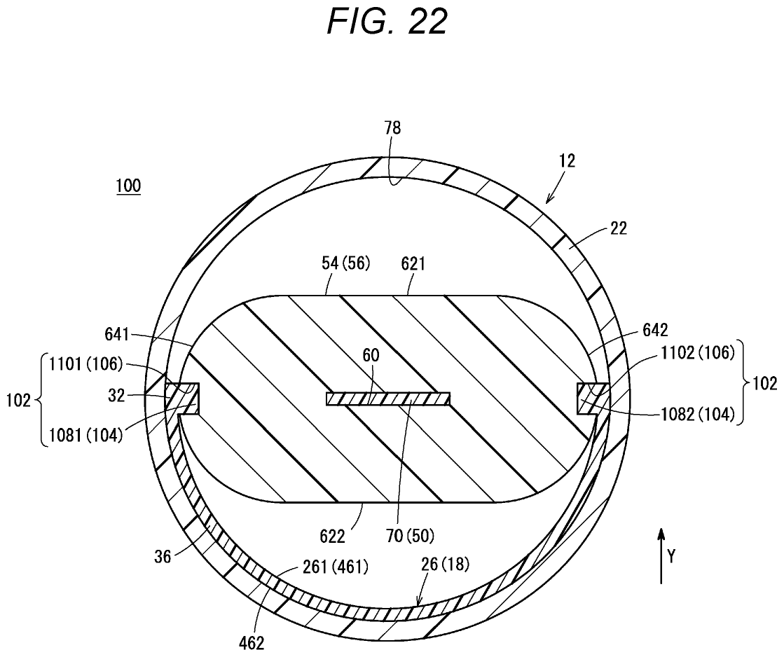

As illustrated in FIG. 22, a transfer instrument 100 according to a first modification includes a movement regulation portion 102. Note that, in the present modification, the same components as those of the above-described transfer instrument 10 are denoted by the same reference numerals, and the detailed description of the same components will be omitted. The same applies to transfer instruments 120, 130, 150, and 160 according to second to fifth modifications described later.

The movement regulation portion 102 includes a first fitting portion 104 provided to the first support portion 26 and a second fitting portion 106 provided to the carrier holding portion 54 at the distal portion of the second shaft 48.

The first fitting portion 104 includes a pair of first projections 1081, 1082 protruding upward from the first support surface 261 of the first support portion 26. The first projections 1081, 1082 each are disposed at equal distances from a center of the first support portion 26 in a width direction. When viewed from a direction orthogonal to the width direction of first support portion 26, cross sections of the first projections 1081, 1082 are rectangular, for example. The cross sections of the first projections 1081, 1082 may have other shapes (semicircular shapes or the like).

The second fitting portion 106 is disposed at each of both end portions in a width direction (convex-shaped portions 641, 642) of the carrier holding portion 54 (pressure-application portion 56). The second fitting portion 106 includes recesses 1101, 1102 recessed from outer surfaces of the convex-shaped portions 641, 642. The recesses 1101, 1102 are recessed in a width direction from the outer surfaces of the convex-shaped portions 641, 642. When viewed from an axial direction of the carrier holding portion 54, cross sections of the recesses 1101, 1102 have rectangular shapes corresponding to the first projections 1081, 1082. The cross sections of the recesses 1101, 1102 may have other shapes (semicircular or the like).

In a state where the first support portion 26 is retracted into the outer cylinder 22 and the first support portion 26 is curved and deformed, the first projections 1081, 1082 of the first fitting portion 104 constituting the movement regulation portion 102 are respectively fitted into the recesses 1101, 1102 of the second fitting portion 106. As a result, relative displacement between the first support portion 26 and the carrier holding portion 54 is restricted, and accordingly, relative movement of the second shaft 48 in the axial direction with respect to the first shaft 24 can be restricted.

As described above, in the transfer instrument 100 according to the first modification, when the first fitting portion 104 and the second fitting portion 106 in the movement regulation portion 102 are fitted in a state (first position) where the first support portion 26 is retracted in the outer cylinder 22, relative movement in the axial direction between the first shaft 24 including the first support portion 26 and the second shaft 48 is prevented. When fitting between the first fitting portion 104 and the second fitting portion 106 of the movement regulation portion 102 is released in a state where the first support portion 26 protrudes from the distal portion of the outer cylinder 22 (second position), the second shaft 48 becomes movable in the distal end direction (the direction of arrow X1) with respect to the first support portion 26.

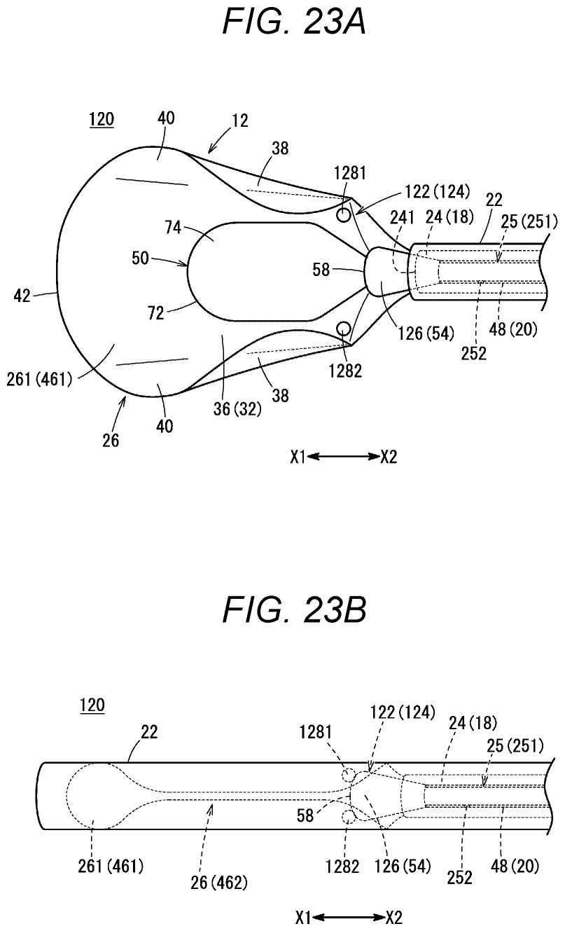

As illustrated in FIG. 23A, a transfer instrument 120 according to the second modification includes a movement regulation portion 122. The movement regulation portion 122 includes a first engagement portion 124 and a second engagement portion 126. The first engagement portion 124 is provided on the front surface 461 of the first support portion 26 including the first support surface 261. The second engagement portion 126 is configured by the carrier holding portion 54 at the distal portion of the second shaft 48.

The first engagement portion 124 includes a pair of second projections 1281 and 1282 projecting upward from the first support surface 261. The second projections 1281 and 1282 each are disposed at equal distances from a center of the first support portion 26 in the width direction. The second engagement portions 126 are disposed on both sides of the distal end of the carrier holding portion 54.

As illustrated in FIG. 23B, in a state where the first support portion 26 is retracted into the outer cylinder 22 and the first support portion 26 is curved and deformed, the second projections 1281 and 1282 of the first engagement portion 124 and the second engagement portion 126 face each other in the axial direction of the outer cylinder 22, and the first engagement portion 124 and the second engagement portion 126 are engaged with each other in the axial direction.

As described above, in the transfer instrument 120 according to the second modification, when the first engagement portion 124 constituting the movement regulation portion 122 and the second engagement portion 126 are engaged in the axial direction in a state (first position) where the first support portion 26 is retracted in the outer cylinder 22 illustrated in FIG. 23B, relative movement in the axial direction between the first shaft 24 including the first support portion 26 and the second shaft 48 is prevented. In a state (second position) where the first support portion 26 protrudes from a distal-end opening 80 of the outer cylinder 22 illustrated in FIG. 23A, the first engagement portion 124 and the second engagement portion 126 do not face each other in the axial direction as the first support portion 26 is unfolded, and the engagement between the first engagement portion 124 and the second engagement portion 126 is released. As a result, the second shaft 48 becomes movable in the distal end direction (the direction of arrow X1) with respect to the first support portion 26.

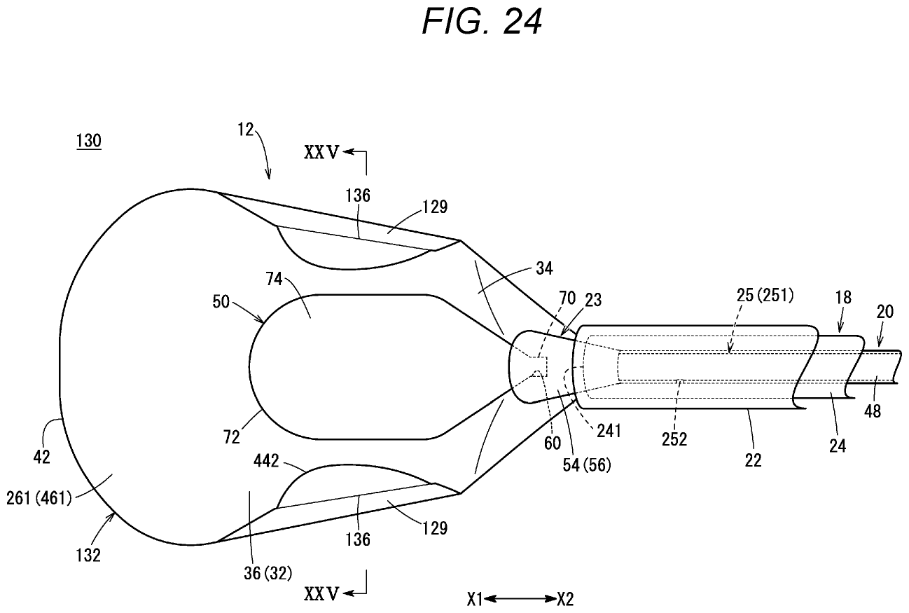

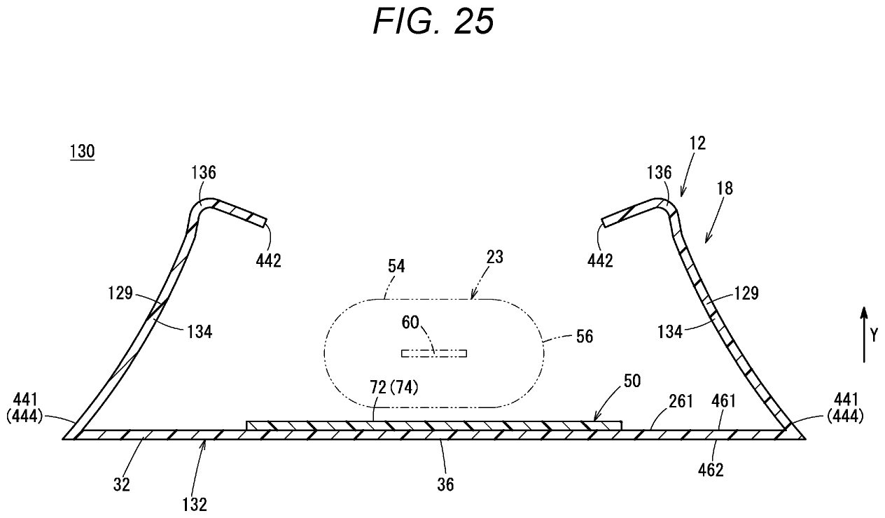

As illustrated in FIGS. 24 and 25, a transfer instrument 130 according to the third modification includes a first support portion 132. Protrusions 129 provided on both sides in a width direction of the first support portion 132 each have an intermediate portion 134 between the free end 442 and the fixed end 441. The intermediate portion 134 has a shape bulging in a convex shape in a direction away from the first support surface 261. The intermediate portion 134 includes an intermediate bent portion 136 bent so as to form a mountain fold on the back surface 462. The intermediate bent portion 136 is disposed closer to the free end 442 in the intermediate portion 134.

As described above, in the transfer instrument 130 according to the third modification, by providing the intermediate bent portion 136 in the protrusion 129 of the first support portion 132, it is possible to easily form the protrusion 129 having the shape bulging in the convex shape in the direction away from the first support surface 261.

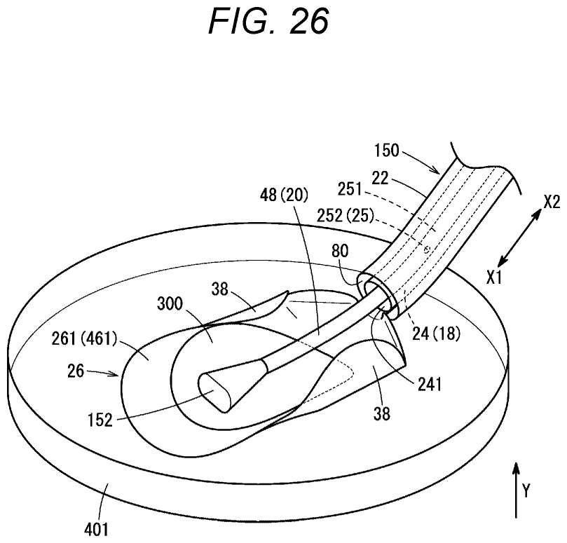

Although the case where the second support portion 50 is attached to the distal portion of the second shaft 48 has been described, the present disclosure is not limited to the second support 50 being attached to the distal portion of the second shaft 48. For example, as in a transfer instrument 150 according to the fourth modification illustrated in FIG. 26, a pressing body 152 may be provided at a distal portion of the second shaft 48. The pressing body 152 has, for example, a flattened cross-sectional shape including a flat bottom surface. The medical sheet 300 placed on the first support surface 261 of the first support portion 26 can be pressed downward by the bottom surface of the pressing body 152 to push out the medical sheet 300 in the distal end direction (the direction of arrow X1) along the first support surface 261 of the first support portion 26.

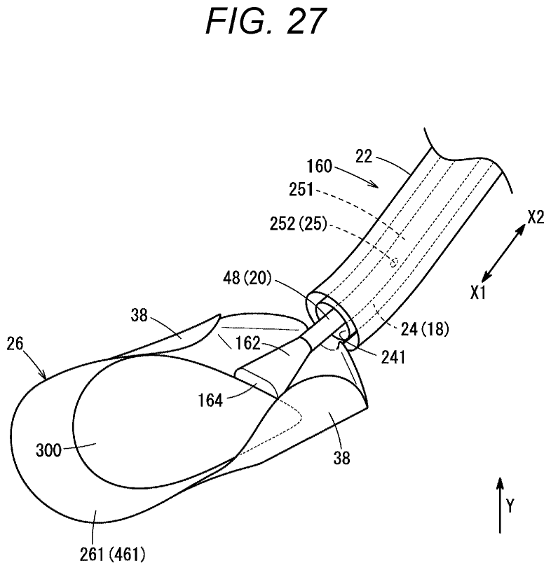

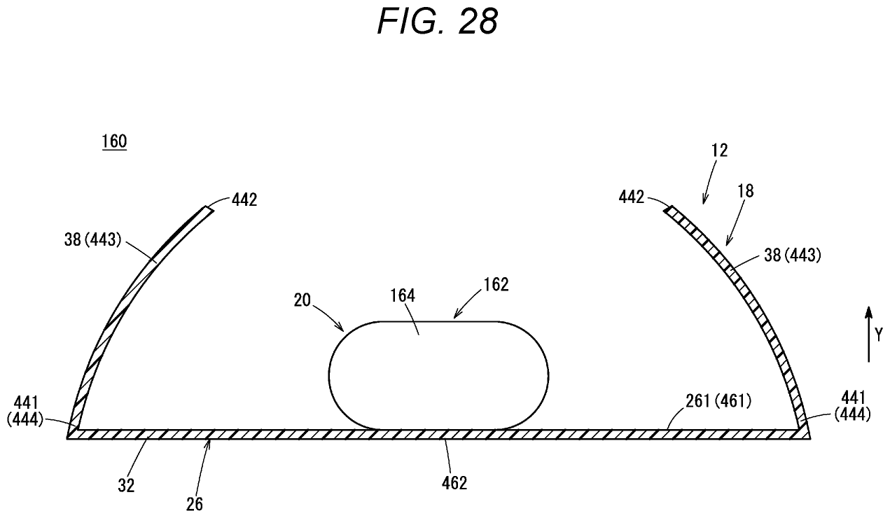

As in a transfer instrument 160 according to the fifth modification illustrated in FIGS. 27 and 28, a pressing body 162 may be provided to the distal portion of the second shaft 48. A distal end of the pressing body 162 includes a pressing surface 164. The pressing surface 164 is a flat surface orthogonal to an axis of the pressing body 162. A proximal end of the pressing body 162 is connected to the distal portion of the second shaft 48. The medical sheet 300 placed on the first support surface 261 of the first support portion 26 can be pressed by the pressing surface 164 of the pressing body 162 to push out the medical sheet 300 in the distal end direction (the direction of arrow X1) along the first support surface 261 of the first support portion 26.

The liquid supply portions in the transfer instruments 100, 120, 130, 150, 160 according to the first to fifth modifications may be any of the liquid flow path and the liquid supply hole disposed in the second shaft 48, the injection port 204 disposed on the outer periphery of the first shaft 24, and the liquid feeding tube 212 disposed on the outer periphery of the second shaft 48.

The detailed description above describes embodiments of a transfer instrument for transferring a medical sheet to a treatment site of a living body. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A transfer instrument for transferring a medical sheet to a treatment site of a living body, the transfer instrument comprising:
an outer cylinder;
a first carrier member including a first shaft that has a tubular shape, extends in an axial direction of the outer cylinder, and is disposed inside the outer cylinder to be movable along the axial direction, and a support portion that has a sheet shape, is disposed at a distal portion of the first shaft, and includes a support surface configured to hold the medical sheet when the distal portion of the first shaft is disposed in a distal end direction with respect to a distal portion of the outer cylinder;
a second carrier member including a second shaft inserted through the first shaft, the second shaft extending along the first shaft and provided movably along the first shaft; and
a liquid supply portion configured to supply a liquid into the first shaft in order to supply the liquid toward the support surface through a distal-end opening of the first shaft, wherein
by moving the first shaft and the second shaft in a proximal end direction with respect to the outer cylinder such that the support portion is retracted into the outer cylinder, the support portion is retracted into the outer cylinder in a state of being curved and deformed, and
by moving the first shaft and the second shaft in the distal end direction with respect to the outer cylinder such that the support portion protrudes from a distal-end opening of the outer cylinder, the support portion is unfolded by being exposed from the outer cylinder in the distal end direction.

2. The transfer instrument according to claim 1, wherein the liquid supply portion includes:
a liquid flow path formed inside the second shaft and extending in an axial direction of the second shaft, and
a liquid supply hole that communicates with the liquid flow path and opens inside the first shaft to an outer peripheral surface of the second shaft.

3. The transfer instrument according to claim 1, wherein
the liquid supply portion includes a liquid feeding tube disposed between an outer peripheral surface of the second shaft and the first shaft; and
the liquid feeding tube includes a liquid outflow port that opens in the first shaft.

4. The transfer instrument according to claim 3, wherein
the liquid feeding tube is fixed to an outer peripheral surface of the second shaft;
the first shaft includes a fitting portion to which the liquid feeding tube is fitted; and
the fitting portion regulates displacement of the liquid feeding tube with respect to the first shaft in a circumferential direction of the first shaft.

5. The transfer instrument according to claim 1, wherein
the first carrier member includes an injection port provided to a portion of the first shaft protruding in a proximal end direction from a proximal end of the outer cylinder, the injection port communicating with an inside of the first shaft.

6. The transfer instrument according to claim 1, wherein
the support portion is formed of a flexible sheet having a front surface including the support surface and a back surface that is a surface opposite to the front surface;
the support portion includes a pair of protrusions protruding upward from both sides in a width direction of the support surface orthogonal to a moving direction of the first shaft; and
in a state where the support portion is retracted in the outer cylinder, the support portion is retracted into the outer cylinder in a state of being curved and deformed such that both sides in a width direction of the support portion on the back surface are in contact with each other.

7. The transfer instrument according to claim 6, wherein
the liquid is supplied to the liquid supply portion in a state where the support portion is retracted in the outer cylinder.

8. A transfer instrument for transferring a medical sheet to a treatment site of a living body, the transfer instrument comprising:
an outer cylinder;
a first carrier member including a first shaft, the first shaft extending in an axial direction of the outer cylinder, and is disposed inside the outer cylinder to be movable along the axial direction, and a support portion disposed at a distal portion of the first shaft, and includes a support surface configured to hold the medical sheet when the distal portion of the first shaft is disposed in a distal end direction with respect to a distal portion of the outer cylinder;
a second carrier member including a second shaft inserted through the first shaft, the second shaft extending along the first shaft and provided movably along the first shaft; and
a liquid supply portion configured to supply a liquid into the first shaft in order to supply the liquid toward the support surface through a distal-end opening of the first shaft.

9. The transfer instrument according to claim 8, wherein
by moving the first shaft and the second shaft in a proximal end direction with respect to the outer cylinder such that the support portion is retracted into the outer cylinder, the support portion is retracted into the outer cylinder in a state of being curved and deformed.

10. The transfer instrument according to claim 8, wherein
by moving the first shaft and the second shaft in the distal end direction with respect to the outer cylinder such that the support portion protrudes from a distal-end opening of the outer cylinder, the support portion is unfolded by being exposed from the outer cylinder in the distal end direction.

11. The transfer instrument according to claim 8, wherein the liquid supply portion includes:
a liquid flow path formed inside the second shaft and extending in an axial direction of the second shaft, and
a liquid supply hole that communicates with the liquid flow path and opens inside the first shaft to an outer peripheral surface of the second shaft.

12. The transfer instrument according to claim 8, wherein
the liquid supply portion includes a liquid feeding tube disposed between an outer peripheral surface of the second shaft and the first shaft; and
the liquid feeding tube includes a liquid outflow port that opens in the first shaft.

13. The transfer instrument according to claim 12, wherein
the liquid feeding tube is fixed to an outer peripheral surface of the second shaft;
the first shaft includes a fitting portion to which the liquid feeding tube is fitted; and
the fitting portion regulates displacement of the liquid feeding tube with respect to the first shaft in a circumferential direction of the first shaft.

14. The transfer instrument according to claim 8, wherein
the first carrier member includes an injection port provided to a portion of the first shaft protruding in a proximal end direction from a proximal end of the outer cylinder, the injection port communicating with an inside of the first shaft.

15. The transfer instrument according to claim 8, wherein
the support portion is formed of a flexible sheet having a front surface including the support surface and a back surface that is a surface opposite to the front surface;
the support portion includes a pair of protrusions protruding upward from both sides in a width direction of the support surface orthogonal to a moving direction of the first shaft; and
in a state where the support portion is retracted in the outer cylinder, the support portion is retracted into the outer cylinder in a state of being curved and deformed such that both sides in a width direction of the support portion on the back surface are in contact with each other.

16. The transfer instrument according to claim 15, wherein
the liquid is supplied to the liquid supply portion in a state where the support portion is retracted in the outer cylinder.

17. A method of transferring a medical sheet to a treatment site of a living body, the method comprising:
placing a transfer instrument for transferring the medical sheet to the treatment site of the living body in an initial state, the transfer instrument including an outer cylinder, a first carrier member including a first shaft, the first shaft extending in an axial direction of the outer cylinder, and is disposed inside the outer cylinder to be movable along the axial direction, and a first support portion disposed at a distal portion of the first shaft, a second carrier member including a second shaft inserted through the first shaft, the second shaft extending along the first shaft and provided movably along the first shaft and includes a second support portion disposed at a distal portion of the second shaft, and a liquid supply portion configured to supply a liquid into the first shaft in order to supply the liquid toward the first support surfaces through a distal-end opening of the first shaft, and wherein in the initial state, the first and second shafts are moved in a distal end direction relative to the outer cylinder so as to be at a protruding position where the first support portion and the second support portion protrude from the distal-end opening of the outer cylinder in the distal end direction;
placing the medical sheet on the second support portion;
retracting the medical sheet into the outer cylinder together with the first support portion and the second support portion from the initial position to a retracted position;
inserting the transfer instrument inserted into the living body and positioning a distal end of the transfer instrument near the treatment site of the living body;
moving the first shaft and the second shaft in a distal end direction relative to the outer cylinder, and unfolding the first support portion, the second support portion and the medical sheet; and
moving the second carrier member in the distal end direction relative to the first carrier member so that the second support portion on which the medical sheet is placed is moved from the retracted position to an advanced position, and wherein the second support portion protrudes in the distal end direction relative to the distal end of the first support portion to move the medical sheet to above the treatment site of the living body and bringing an overhanging portion of the medical sheet into contact with the treatment site of the living body.

18. The method according to claim 17, further comprising:
connecting the liquid supply instrument to a hub of the second carrier member and supplying supply a liquid to the liquid flow path, which is discharged from the liquid supply hole to radially outward of the second shaft, and flows toward the distal-end opening of the first shaft along an axial direction of the first and second shafts through a space between an inner peripheral surface of the first shaft and an outer peripheral surface of the second shaft; and
stopping the supply of the liquid to the first support portion after a predetermined amount of liquid is supplied to the first support surface of the first support portion.

19. The method according to claim 17, further comprising:
moving the second carrier member from the second position to the first position to withdraw the second support portion from between the treatment site and the medical sheet; and
withdrawing the transfer instrument from the living body with the first support portion and the second support portion retracted into the outer cylinder.

20. The method according to claim 17, wherein the treatment site is a heart of the living body.
